SEQUENCE LISTING (11 sequences)

SEQ ID NO: 1 - protein, 124 aa, Artificial Sequence
  source: /note=“Description of Artificial Sequence Synthetic polypeptide”
QVQLVESGGGVVQPGRSLRLSCAASGFAFSSYGMHWVRQAPGKGLEWVAVIWFDGTKKYYTDSVKGRFTISRDNSKNTLYLQMNTLRAEDTAVYYCARDRGIGARRGPYYMDVWGKGTTVTVSS

SEQ ID NO: 2 - protein, 108 aa, Artificial Sequence
  source: /note=“Description of Artificial Sequence Synthetic polypeptide”
DIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGGGTKVEIKR

SEQ ID NO: 3 - protein, 5 aa, Artificial Sequence
  source: /note=“Description of Artificial Sequence Synthetic peptide”
SYGMH

SEQ ID NO: 4 - protein, 17 aa, Artificial Sequence
  source: /note=“Description of Artificial Sequence Synthetic peptide”
VIWFDGTKKYYTDSVKG

SEQ ID NO: 5 - protein, 15 aa, Artificial Sequence
  source: /note=“Description of Artificial Sequence Synthetic peptide”
DRGIGARRGPYYMDV

SEQ ID NO: 6 - protein, 11 aa, Artificial Sequence
  source: /note=“Description of Artificial Sequence Synthetic peptide”
RASQSISSYLN

SEQ ID NO: 7 - protein, 7 aa, Artificial Sequence
  source: /note=“Description of Artificial Sequence Synthetic peptide”
AASSLQS

SEQ ID NO: 8 - protein, 9 aa, Artificial Sequence
  source: /note=“Description of Artificial Sequence Synthetic peptide”
QQSYSTPLT

SEQ ID NO: 9 - protein, 10 aa, Artificial Sequence
  source: /note=“Description of Artificial Sequence Synthetic peptide”
GFAFSSYGMH

SEQ ID NO: 10 - protein, 453 aa, Artificial Sequence
  source: /note=“Description of Artificial Sequence Synthetic polypeptide”
QVQLVESGGGVVQPGRSLRLSCAASGFAFSSYGMHWVRQAPGKGLEWVAVIWFDGTKKYYTDSVKGRFTISRDNSKNTLYLQMNTLRAEDTAVYYCARDRGIGARRGPYYMDVWGKGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 11 - protein, 214 aa, Artificial Sequence
  source: /note=“Description of Artificial Sequence Synthetic polypeptide”
DIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

(19) United States
(12) Patent Application Publication
(10) Pub. No.: US 2022/0372123 A1
(43) Pub. Date: Nov. 24, 2022

(54) ANTI-BETA-AMYLOID ANTIBODY FOR TREATING ALZHEIMER'S DISEASE

(71) Applicants: Biogen MA Inc., Cambridge, MA (US); Neurimmune AG, Schlieren (CH)

(72) Inventors: Samantha Budd Haeberlein, Cambridge, MA (US); Ying Tian, Cambridge, MA (US); Laura Nisenbaum, Cambridge, MA (US); Raj Rajagovindan, Cambridge, MA (US); Gersham Dent, Cambridge, MA (US); John Beaver, Cambridge, MA (US); Edward Plowey, Cambridge, MA (US); Thierry Bussiere, Cambridge, MA (US); Roger Nitsch, Zumikon (CH)

(21) Appl. No.: 17/770,997

(22) PCT Filed: Oct. 21, 2020
(86) PCT No.: PCT/US20/56676

§ 371 Date: Apr. 21, 2022

Related U.S. Application Data
(60) Provisional application No. 62/924,633, filed on Oct. 22, 2019.

(51) Int. Cl. C07K 16/18; A61P 25/28

(52) U.S. Cl. CPC C07K 16/18; A61P 25/28; A61K 2039/505

(57) ABSTRACT

Provided are methods for treating Alzheimer's disease in a human subject in need thereof comprising administration of multiple doses of an anti-beta-amyloid antibody (e.g., aducanumab) to the subject.

### CROSS-REFERENCE TO RELATED APPLICATIONS

This application claims the benefit of priority of U.S. Provisional Appl. No. 62/924,633, filed Oct. 22, 2019, the contents of which are incorporated by reference herein in their entirety.

### SEQUENCE LISTING

This application contains a Sequence Listing which has been submitted electronically in ASCII format and is hereby incorporated by reference in its entirety. Said ASCII copy, created on Oct. 16, 2020, is named 13751-0323WO1_SL.txt and is 10,440 bytes in size.

### TECHNICAL FIELD

This disclosure relates generally to methods for treating Alzheimer's disease.

### BACKGROUND

Alzheimer's disease (AD) is a progressive neurodegenerative disorder clinically characterized by cognitive impairment, behavioral disturbances, psychiatric symptoms, and disability in activities of daily living. These clinical manifestations constitute AD dementia.

AD International estimates that the number of people living with dementia worldwide will increase from the current value of 47 million to 131 million by 2050. Being the most common cause of dementia, AD accounts for 60 to 80% of dementia cases. In the United States, it is estimated that 5.2 million Americans suffer from dementia caused by AD, and that by 2050 the prevalence will double or triple unless an effective treatment is found.

Clinical research criteria for dementia due to AD have been recently updated and conforming to the current concept of the disease, a diagnostic framework was developed to embrace pre-dementia stages of AD (e.g., prodromal AD). The main neuropathological hallmarks of the disease are (i) extracellular senile (neuritic) plaques containing aggregated β-amyloid (Aβ) peptides and (ii) intraneuronal neurofibrillary tangles (NFTs) composed of abnormal hyperphosphorylated Tau protein. The “amyloid cascade” hypothesis proposes that the driving force behind the disease process is the accumulation of Aβ resulting from an imbalance between Aβ production and Aβ clearance in the brain.

Aβ is a peptide generated from the metabolism of amyloid precursor protein. Several Aβ peptide alloforms exist (e.g., Aβ40, Aβ42). These monomeric peptides have a variable tendency to aggregate into higher order dimers and oligomers. Through a process of fibrillogenesis, soluble oligomers may transition into insoluble deposits having β pleated sheet structure. These deposits are also referred to as amyloid plaques and are composed of predominantly fibrillar amyloid. Both soluble and fibrillar forms of Aβ appear to contribute to the disease process.

Biomarker, clinicopathologic, and cohort studies suggest that the disease process commences 10 to 20 years before the clinical onset of symptoms, and some of the early pathological findings include the deposition of neocortical neuritic plaques and mesial temporal NFTs followed years later by neocortical NFTs.

Thus, there is a need for methods to treat Alzheimer's disease patients.

### SUMMARY

This disclosure fulfills the need for methods to treat Alzheimer's disease (AD) patients.

In one aspect, the disclosure features a method for treating Alzheimer's disease in a human subject in need thereof. The method involves administering to the human subject multiple doses of an anti-beta-amyloid antibody, wherein the multiple doses are administered as follows: (a) administering the anti-beta-amyloid antibody to the subject in an amount of 1 mg/kg of body weight of the subject; (b) 4 weeks after step (a), administering the antibody to the subject in an amount of 1 mg/kg of body weight of the subject; (c) 4 weeks after step (b), administering the antibody to the subject in an amount of 3 mg/kg of body weight of the subject; (d) 4 weeks after step (c), administering the antibody to the subject in an amount of 3 mg/kg of body weight of the subject; (e) 4 weeks after step (d), administering the antibody to the subject in an amount of 6 mg/kg of body weight of the subject; (f) 4 weeks after step (e), administering the antibody to the subject in an amount of 6 mg/kg of body weight of the subject; and (g) in consecutive intervals of 4 weeks after step (f), administering at least 15 doses of the antibody in an amount of 10 mg/kg of body weight of the subject, wherein the anti-beta-amyloid antibody comprises a heavy chain variable region (VH) and a light chain variable region (VL), wherein the VH comprises a complementarity determining region (VHCDR1) with the amino acid sequence of SEQ ID NO:3, a VHCDR2 with the amino acid sequence of SEQ ID NO:4, and a VHCDR3 with the amino acid sequence of SEQ ID NO:5, and wherein the VL comprises a VLCDR1 with the amino acid sequence of SEQ ID NO:6, a VLCDR2 with the amino acid sequence of SEQ ID NO:7, and a VLCDR3 with the amino acid sequence of SEQ ID NO:8.

In some embodiments, step (g) comprises administering at least 16 doses, at least 17 doses, at least 18 doses, at least 19 doses, or at least 20 doses of the antibody, in consecutive intervals of 4 weeks, each in an amount of 10 mg/kg of body weight of the subject.

In some embodiments of any of the foregoing methods, all of the doses specified in steps (a)-(g) are administered without interruption even if the human subject develops an Amyloid

Related Imaging Abnormality (ARIA) during the course of treatment.

In some embodiments of any of the foregoing methods, the human subject develops an ARIA during the course of treatment and all of the doses specified in steps (a)-(g) are administered without interruption.

In some embodiments of any of the foregoing methods, the Alzheimer's disease is mild Alzheimer's disease, early Alzheimer's disease, prodromal Alzheimer's disease, mild Alzheimer's disease dementia, or mild cognitive impairment due to Alzheimer's disease.

In some embodiments of any of the foregoing methods, each administration is performed intravenously.

In some embodiments of any of the foregoing methods, the human subject is confirmed to have a brain amyloid beta pathology prior to the initiation of treatment. In some embodiments, the brain amyloid beta pathology is determined by positron emission tomography (PET) imaging. In some embodiments, the brain amyloid beta pathology is determined by Congo red staining and birefringence under polarized microscopy. In some embodiments, the brain amyloid beta pathology is determined by immunohistochemistry. In some embodiments, the brain amyloid beta pathology is determined by electron microscopy or mass spectrometry. In some embodiments, the brain amyloid beta pathology is determined by CSF analysis. In some embodiments, the brain amyloid beta pathology is determined by blood analysis.

In another aspect, the disclosure features a method for treating mild Alzheimer's disease, early Alzheimer's disease, prodromal Alzheimer's disease, mild Alzheimer's disease dementia, or mild cognitive iwairment due to Alzheimer's disease in a human subject in need thereof. The method involves administering to the human subject multiple doses of an anti-beta-amyloid antibody, wherein the method comprises administering in consecutive intervals of 4 weeks at least 6 doses of the antibody, wherein each dose is in an amount of 10 mg/kg of body weight of the subject, wherein the anti-beta-amyloid antibody comprises a heavy chain variable region (VH) and a light chain variable region (VL), wherein the VH comprises a complementarity determining region (VHCDRI) with the amino acid sequence of SEQ ID NO:3, a VHCDR2 with the amino acid sequence of SEQ ID NO:4, and a VHCDR3 with the amino acid sequence of SEQ ID NO:5, and wherein the VL comprises a VLCDR1 with the amino acid sequence of SEQ ID NO:6, a VLCDR2 with the amino acid sequence of SEQ ID NO:7, and a VLCDR3 with the amino acid sequence of SEQ ID NO:8.

In some embodiments, the method comprises administering in consecutive intervals of 4 weeks at least 8 doses, at least 9 doses, at least 10 doses, at least 11 doses, at least 12 doses, at least 13 doses, at least 14 doses, at least 15 doses, at least 16 doses, at least 17 doses, at least 18 doses, at least 19 doses, or at least 20 doses of the antibody, wherein each dose is in an amount of 10 mg/kg of body weight of the subject.

In some embodiments of any of the foregoing methods, all of the doses specified are administered without interruption even if the human subject develops an ARIA during the course of treatment.

In some embodiments of any of the foregoing methods, the human subject develops an ARIA during the course of treatment and all of the doses specified are administered without interruption.

In some embodiments of any of the foregoing methods, each administration is performed intravenously.

In some embodiments of any of the foregoing methods, the human subject is confirmed to have a brain amyloid beta pathology prior to the initiation of treatment. In some embodiments, the brain amyloid beta pathology is determined by PET imaging.

In another aspect, the disclosure features a method for treating Alzheimer's disease in a human subject in need thereof. The method involves administering to the human subject multiple doses of an anti-beta-amyloid antibody, wherein the multiple doses are administered as follows: (a) administering the anti-beta-amyloid antibody to the subject in an amount of 1 mg/kg of body weight of the subject; (b) 4 weeks after step (a), administering the antibody to the subiect in an amount of 1 mg/kg of body weight of the subject; (c) 4 weeks after step (b), administering the antibody to the subject in an amount of 3 mg/kg of body weight of the subject; (d) 4 weeks after step (c), administering the antibody to the subject in an amount of 3 mg/kg of body weight of the subject; (e) 4 weeks after step (d), administering the antibody to the subject in an amount of 6 mg/kg of body weight of the subject; (f) 4 weeks after step (e), administering the antibody to the subject in an amount of 6 mg/kg of body weight of the subject; and (g) in consecutive intervals of 4 weeks after step (f), administering the antibody to the subject in an amount of 10 mg/kg of body weight of the subject, wherein the anti-beta-amyloid antibody comprises a heavy chain variable region (VH) and a light chain variable region (VL), wherein the VH comprises a complementarity determining region (VHCDR1) with the amino acid sequence of SEQ ID NO:3, a VHCDR2 with the amino acid sequence of SEQ ID NO:4, and a VHCDR3 with the amino acid sequence of SEQ ID NO:5, wherein the VL comprises a VLCDR1 with the amino acid sequence of SEQ ID NO:6, a VLCDR2 with the amino acid sequence of SEQ ID NO:7, and a VLCDR3 with the amino acid sequence of SEQ ID NO:8, and wherein all of the doses specified are administered without interruption even if the human subject develops an ARIA during the course of treatment.

In some embodiments, the human subject develops an ARIA during the course of treatment and all of the doses specified are administered without interruption.

In some embodiments of any of the foregoing methods, step (g) comprises administering at least 6 doses, at least 7 doses, at least 8 doses, at least 9 doses, at least 10 doses, at least 11 doses, at least 12 doses, at least 13 doses, at least 14 doses, at least 15 doses, at least 16 doses, at least 17 doses, at least 18 doses, at least 19 doses, or at least 20 doses of the antibody, in consecutive intervals of 4 weeks, each in an amount of 10 mg/kg of body weight of the subject.

In some embodiments of any of the foregoing methods, the Alzheimer's disease is mild Alzheimer's disease, early Alzheimer's disease, prodromal Alzheimer's disease, mild Alzheimer's disease dementia, or mild cognitive impairment due to Alzheimer's disease.

In some embodiments of any of the foregoing methods, each administration is performed intravenously.

In some embodiments of any of the foregoing methods, the human subject is confirmed to have a brain amyloid beta pathology prior to the initiation of treatment. In some embodiments. the brain amyloid beta pathology is determined by PET imaging.

In another aspect, the disclosure features a method for treating Alzheimer's disease in a human subject in need thereof. The method involves administering to the human subject multiple doses of an anti-beta-amyloid antibody, wherein the multiple doses are administered as follows: (a) administering intravenously the anti-beta-amyloid antibody to the subject in an amount of 1 mg/kg of body weight of the subject; (b) 4 weeks after step (a), administering intravenously the antibody to the subject in an amount of 1 mg/kg of body weight of the subject; (c) 4 weeks after step (b), administering intravenously the antibody to the subject in an amount of 3 mg/kg of body weight of the subject; (d) 4 weeks after step (c), administering intravenously the antibody to the subject in an amount of 3 mg/kg of body weight of the subject; (e) 4 weeks after step (d), administering intravenously the antibody to the subject in an amount of 6 mg/kg of body weight of the subject; (f) 4 weeks after step (e), administering intravenously the antibody to the subject in an amount of 6 mg/kg of body weight of the subject; and (g) in consecutive intervals of 4 weeks after step (f), administering intravenously at least 6 doses of the antibody in an amount of 10 mg/kg of body weight of the subject, wherein the anti-beta-amyloid antibody comprises a heavy chain variable region (VH) and a light chain variable region (VL), wherein the VH comprises a complementarity determining region (VHCDR1) with the amino acid sequence of SEQ ID NO:3, a VHCDR2 with the amino acid sequence of SEQ ID NO:4, and a VHCDR3 with the amino acid sequence of SEQ ID NO:5, and wherein the VL comprises a VLCDR1 with the amino acid sequence of SEQ ID NO:6, a VLCDR2 with the amino acid sequence of SEQ ID NO:7, and a VLCDR3 with the amino acid sequence of SEQ ID NO:8.

In some embodiments, step (g) comprises administering intravenously at least 7 doses, at least 8 doses, at least 9 doses, at least 10 doses, at least 11 doses, at least 12 doses, at least 13 doses, at least 14 doses, at least 15 doses, at least 16 doses, at least 17 doses, at least 18 doses, at least 19 doses, or at least 20 doses of the antibody, in consecutive intervals of 4 weeks, each in an amount of 10 mg/kg of body weight of the subject.

In some embodiments of any of the foregoing methods, all of the doses specified in steps (a)-(g) are administered without interruption even if the human subject develops an ARIA during the course of treatment.

In some embodiments of any of the foregoing methods, the human subject develops an ARIA during the course of treatment and all of the doses specified in steps (a)-(g) are administered without interrpotion.

In some embodiments of any of the foregoing methods, the Alzheimer's disease is mild Alzheimer's disease, early Alzheimer's disease, prodromal Alzheimer's disease, mild Alzheimer's disease dementia, or mild cognitive impairment due to Alzheimer's disease.

In some embodiments of any of the foregoing methods, the human subject is confirmed to have a brain amyloid beta pathology prior to the initiation of treatment. In some embodiments, the brain amyloid beta pathology is determined by PET imaging.

In some embodiments of any of the methods described herein, the VH of the anti-beta-amyloid antibody comprises the amino acid sequence of SEQ ID NO:1, and the VL of the anti-beta-amyloid antibody comprises the amino acid sequence of SEQ ID NO:2.

In some embodiments of any of the methods described herein, the anti-beta-amyloid antibody comprises a human IgG1 constant region.

In some embodiments of any of the methods described herein, the anti-beta-amyloid antibody comprises a heavy chain and a light chain, wherein the heavy chain comprises the amino acid sequence of SEQ ID NO:10, and the light chain comprises the amino acid sequence of SEQ ID NO:11.

In another aspect, the disclosure features a method for reducing Abeta (Aβ), in particular Aβ plaque and tau in a human subject in need thereof. The method involves administering to the human subject multiple doses of an anti-beta-amyloid antibody, wherein the anti-beta-amyloid antibody comprises a heavy chain variable region (VH) and a light chain variable region (VL), wherein the VH comprises a complementarity determining region (VHCDR1) with the amino acid sequence of SEQ ID NO:3, a VHCDR2 with the amino acid sequence of SEQ ID NO:4, and a VHCDR3 with the amino acid sequence of SEQ ID NO:5, and wherein the VL comprises a VLCDR1 with the amino acid sequence of SEQ ID NO:6, a VLCDR2 with the amino acid sequence of SEQ ID NO:7, and a VLCDR3 with the amino acid sequence of SEQ ID NO:8. In a particularly preferred embodiment, the antibody is BIIB037, also known as aducanumab; see infra.

In this aspect, the invention is inter alia based on the observation of aducanumab-induced amyloid removal, and a previously unrecognized pleiotropic activity of aducanumab on pathological tau variants in human patients.

As illustrated in Example 14 a human patient in the PRIME trial, who had received 30 subsequent 6 mg/kg doses of aducanumak (LTE patient). besides showine amyloid removal indicated by the PET data (FIGS. 10B and 10C) and unusual “moth-eaten” amyloid plaque structure (FIGS. 11D and 12B) as well as microglial plaque engagement (FIGS. 12C and 12D) surprisingly also showed pTau neuropathology reduction as evidenced by lower neocortical pTau density in the LTE patient compared to a range of untreated HIGH AD cases (FIG. 13).

Surprisingly, the treatment effect did not reverse the special sequence of appearance of tau pathologies according to a “reverse Braak pattern” but reduced the intensity of tau staining in all Braak V-VI regions. Such pattern of reduced tau staining intensity in all Braak V-VI regions can only be explained by a reduction in the amount of pathological tau in all Braak regions that had previously built up tau pathology according to the known special progression of the disease. In other words: a treatment directed against tau as the therapeutic target would be expected to reduce tau pathologies in all affected Braak regions, but not “reverse” the pattern of Braak staging to an earlier stage, e.g. from stage VI back to stage II.

Aducanumab treatment showed this very pattern of reducing the amount of pathologic tau staining in all previously affected Braak regions. The mechanism is surprising because the well-cited “amyloid cascade hypothesis” posits amyloid as a trigger of tau pathology. Following this logic, aducanumab-induced amyloid removal would stop or lower the formation of “new” tau pathology.

Taking the data and possible theory behind into account, the Phase 3 studies, Study 1 and Study 2 have been assessed regarding the effect of the treatment with aducanumab on tau pathology and clinical benefit of the patients. Hence, as illustrated in Example 12 and shown in FIG. 9, PET and CSF biomarker studies showed that aducanumab reduced Aβ tau pathology and neurodegeneration in participants with early Alzheimer's disease.

The observed effect, however, indicates not only removal of “new” tau pathology, but more importantly the reduction of pre-existing tau pathology via a novel therapeutic mechanism that is not covered by the logic of the amyloid cascade hypothesis. Without intending to be bound by theory, this novel mechanism could include proteasome-mediated degradation of intracellular pathological tau within affected neurons, the removal of extracellular species of pathological tau vial phagocytosis of microglial cells or macrophages, potentially explaining the observed decreases in CSF levels of pathological phospho-tau in aducanumab-treated patients.

If pathological tau is subject to natural turnover, blocking its production could also lead to the observed reduced staining intensities in the aducanumab-treated patient. Such reductions in the formation of pathological could be explained by aducanumab-induced neutralization and removal of neurotoxic oligomeric amyloid beta species from pre-synaptic axonal terminals, thereby protecting axons from amyloid damage resulting in remaining unphosphorylated microtubule-bound tau within its physiological axonal localization, thereby preventing abnormal re-localization of tau from axonal to somato-dendritic compartments in amyloid affected neurons, where tau gets abnormally phosphorylated and becomes aggregation-prone. Natural mechanisms for the turnover of tau include proteasomal degradation following ubiquitination, microglial phagocytosis and perivascular drainage into CSF and blood.

A further surprising and unexpected possibility is pleiotropy of aducanumab's function as a stimulator of microglial phagocytosis. Such pleiotropic effects could be explained by a previously unknown activity of aducanumab on pathologic, aggregated species of tau targeting these for microglial phagocytosis and degradation. It may also involve uptake and degradation of previously unknown co-aggregates of amyloid beta oligomers with pathological tau aggregates. Such co-aggregation of amyloid beta aggregates with other unrelated proteins is known. As an example, amyloid beta co-aggregates with islet amyloid polypeptide IAPP (amylin), a self-aggregating polypeptide produced by insulin-secreting pancreatic beta cells.

Altogether, the data of the clinical trials performed in accordance with the present invention led to the conclusion that aducanumab induced amyloid removal causes not only an attenuation of disease-related increases in tau, but reductions in pathological forms of tau in brains of patients with Alzheimer's disease, associated with the cognitive improvement of the treated patients.

This is a totally unexpected finding due to the prior knowledge that aducanumab binds amyloid but not tau.

Indeed, hitherto when targeting Aβ in the immunotherapy of Alzheimer's disease, tau was only considered as a biomarker in cerebrospinal fluid (CSF) and over the last years, in positron emission tomography (PET) imaging for AD diagnosis, monitoring clinical stage and to sub-classify the type of cognitive decline; see for review, e.g., Gabelli, J. Lab. Precis. Med. 15 (2020) |http://dx.doi.org/10.21037/j1pm.2019.12.04 and Nguyen et al., Diagnostics 2020, 10, 326; doi:10.3390/diagnostics10050326.

Accordingly, in one embodiment the present invention features a method of treating Alzheimer's disease in a human subiect in need thereof, the method comprising administering to the human subject a therapeutically effective amount of an anti-beta-amyloid antibody comprising a heavy chain variable region (VH) and a light chain variable region (VL), wherein the VH comprises a VH complementarity determining region 1 (VHCDR1) with the amino acid sequence of SEQ ID NO:3, a VHCDR2 with the amino acid sequence of SEQ ID NO:4, and a VHCDR3 with the amino acid sequence of SEQ ID NO:5, and wherein the VL comprises a

VLCDRI with the amino acid sequence of SEQ ID NO:6, a VLCDR2 with the amino acid sequence of SEQ ID NO:7, and a VLCDR3 with the amino acid sequence of SEQ ID NO:8, wherein the human subject has p-tau tangles, p-tau threads, and/or p-tau neuritic plaques. In some instances, the human subject has neocortical p-tau tangles, neocortical p-tau threads, and/or neocortical p-tau neuritic plaques.

In some embodiments, the administration of the anti-beta-amyloid antibody reduces p-tau tangles, p-tau threads, and/or p-tau neuritic plaques in the brain of the human subject or the amount of phosphorylated tau (p-tau) and/or total tau (t-tau) in the cerebrospinal fluid (CSF) of the human subject. In certain instances, prior to the administration of the anti-beta-amyloid antibody, p-tau tangles, p-tau threads, and/or p-tau neuritic plaques are detected by positron emission tomography (PET) scanning of the human subject's brain or by analysis of the amount of p-tau and/or t-tau in the human subject's CSF.

In some cases, the method further comprises monitoring during treatment p-tau tangles, p-tau threads, and/or p-tau neuritic plaques by PET scanning of the human subject's brain or by analysis of the amount of p-tau and/or t-tau in the human subject's CSF.

In certain cases, the amount of the anti-beta-amyloid antibody administered and/or frequency of administration of the anti-beta-amyloid antibody is adjusted during treatment by monitoring p-tau tangles, p-tau threads, and/or p-tau neuritic plaques in the human subject's brain using PET scanning, or by analysis of the amount of p-tau and/or t-tau in the human subject's CSF.

In some cases, the treatment results in (i) a reduction in SUVR, density, and/or distribution of p-tau tangles, p-tau threads, and/or p-tau neuritic plaques relative to a prior PET scan, or (ii) a reduction in the amount of p-tau and/or t-tau in a CSF analysis relative to a prior CSF analysis.

In another aspect the disclosure features a method of reducing tau in a human subject in need thereof. The method comprises administeriing to the human subject an effective amount of an anti-beta-amyloid antibody comprising a VH and a VL, wherein the VH comprises a VHCDR1 with the amino acid sequence of SEQ ID NO:3, a VHCDR2 with the amino acid sequence of SEQ ID NO:4, and a VHCDR3 with the amino acid sequence of SEQ ID NO:5, and wherein the VL comprises a VLCDR1 with the amino acid sequence of SEQ ID NO:6, a VLCDR2 with the amino acid sequence of SEQ ID NO:7, and a VLCDR3 with the amino acid sequence of SEQ ID NO:8. In some instances, the human subject has Alzheimer's disease.

In yet another aspect, the disclosure features a method of reducing beta amyloid and tau in a human subject in need thereof. The method comprises administering to the human subject an effective amount of an anti-beta-amyloid antibody comprising a VH and a VL, wherein the VH comprises a VHCDR1 with the amino acid sequence of SEQ ID NO:3, a VHCDR2 with the amino acid sequence of SEQ ID NO:4, and a VHCDR3 with the amino acid sequence of SEQ ID NO:5, and wherein the VL comprises a VLCDR1 with the amino acid sequence of SEQ ID NO:6, a VLCDR2 with the amino acid sequence of SEQ ID NO:7, and a VLCDR3 with the amino acid sequence of SEQ ID NO:8. In some instances, the human subject has Alzheimer's disease.

In another aspect the disclosure provides a method of treating Alzheimer's disease by reducing the amount of tau in a human subject in need thereof. The method comprises administering to the human subject an effective amount of an anti-beta-amyloid antibody comprising a VH and a VL, wherein the VH comprises a VHCDR1 with the amino acid sequence of SEQ ID NO:3, a VHCDR2 with the amino acid sequence of SEQ ID NO:4, and a VHCDR3 with the amino acid sequence of SEQ ID NO:5, and wherein the VL comprises a VLCDR1 with the amino acid sequence of SEQ ID NO:6, a VLCDR2 with the amino acid sequence of SEQ ID NO:7, and a VLCDR3 with the amino acid sequence of SEQ ID NO:8.

In some instances, the human subject is, or has previously been, diagnosed as having p-tau tangles, p-tau threads, and/or p-tau neuritic plaques in the brain and/or an increased amount of p-tau and/or t-tau in the human subject's CSF relative to a human subject without Alzheimer's disease.

In some cases, the amount of tau in the brain and/or the CSF of the human subject is reduced. In certain cases, the amount of p-tau and/or t-tau in the human subject is reduced.

In certain cases, the human subject has elevated levels of tau, prior to administration of the anti-beta-amyloid antibody as measured in the CSF or in the brain by PET scanning. Tau levels are elevated in AD (see e.g., Blennow and Zetterberg, J. Int. Med 2018, Biomarkers for Alzheimer's disease: current status and prospects for the future). p-Tau and t-Tau levels can be measured in CSF (e.g., collected by lumbar puncture) or by blood-based tests.

In some cases, the Alzheimer's disease is mild Alzheimer's disease, early Alzheimer's disease, prodromal Alzheimer's disease, mild Alzheimer's disease dementia, mild cognitive impairment due to Alzheimer's disease, mid-stage Alzheimer's disease, or late-stage Alzheimer's disease, optionally wherein mid-stage Alzheimer's disease is characterized by a Mini-Mental State Examination (MMSE) score of about 10-20 or equivalent score on other scales and late-stage Alzheimer's disease is characterized by an MMSE score of 9 or less or equivalent score on other scales. In some cases, the Alzheimer's disease is mild cognitive impairment due to Alzheimer's disease. In certain cases, the Alzheimer's disease is mild Alzheimer's disease dementia.

In some embodiments, the VH comprises the amino acid sequence of SEQ ID NO:1 and the VL comprises the amino acid sequence of SEQ ID NO:2. In certain instances, the anti-beta- amyloid antibody comprises a heavy chain comprising the amino acid sequence of SEQ ID NO:10 and a light chain comprising the amino acid sequence of SEQ ID NO:11.

In some cases, the anti-beta-amyloid antibody is administered intravenously.

In certain embodiments, the method comprises administering the anti-beta-amyloid antibody in an amount of 3 mg antibody/kg of body weight of the human subject. In certain embodiments, the method comprises administering the anti-beta-amyloid antibody in an amount of 6 mg antibody/kg of body weight of the human subject. In other embodiments, the method comprises administering the anti-beta-amyloid antibody in an amount of 10 mg antibody/kg of body weight of the human subject.

In certain embodiments, the method comprises administering the anti-beta-amyloid antibody in multiple doses as follows:

(a) administering the anti-beta-amyloid antibody to the human subject in an amount of 1 mg antibody/kg of body weight of the human subject;

(b) 4 weeks after step (a), administering the antibody to the human subject in an amount of 1 mg antibody/kg of body weight of the human subject;

(c) 4 weeks after step (b), administering the antibody to the human subject in an amount of 3 mg antibody/kg of body weight of the human subject:

(d) 4 weeks after step (c), administering the antibody to the human subject in an amount of 3 mg antibody/kg of body weight of the human subject;

(e) 4 weeks after step (d), administering the antibody to the human subject in an amount of 6 mg antibody/kg of body weight of the human subject;

(f) 4 weeks after step (e), administering the antibody to the human subject in an amount of 6 mg antibody/kg of body weight of the human subject; and

(g) in consecutive intervals of 4 weeks after step (f), administering the antibody to the human subject in an amount of 10 mg antibody/kg of body weight of the human subject.

In some embodiments, the method comprises administering the antibody at a cumulative dose of at least 150 mg antibody/kg of body weight of the human subject. In certain embodiments, the method comprises administering the antibody at a cumulative dose of at least 200 mg antibody/kg of body weight of the human subject.

In certain embodiments, the method comprises administering the antibody in an amount of 10 mg antibody/kg of body weight of the human subject every 4 weeks over at least 52 weeks. In other embodiments, the method comprises administering the antibody in an amount of 6 mg antibody/kg of body weight of the human subject every 4 weeks over at least 112 weeks.

In some embodiments, the method comprises administering the antibody to the human subject in multiple doses and wherein the multiple doses comprise:

(a) at least two doses of 3 mg antibody/kg of body weight of the human subject every 4 weeks; and

(b) at least 30 doses of 6 mg antibody/kg of body weight of the human subject every 4 weeks.

In certain embodiments, the human subject is an ApoE3 carrier.

In some embodiments, the human subject does not develop an Amyloid Related Imaging Abnormality (ARIA) during the course of treatment that requires suspension of treatment.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, the exemplary methods and materials are described below. All publications, patent applications, patents, and other references mentioned herein are incorporated by reference in their entirety. In case of conflict, the present application, including definitions, will control. The materials, methods, and examples are illustrative only and not intended to be limiting.

Other features and advantages of the invention will be apparent from the following detailed description and from the claims.

For the avoidance of any doubt it is emphasized that the expressions “in some embodiments”, “in a certain embodiments”, “in certain instances”, “in some instances”, “in a further embodiment”, “in one embodiment” and “in a further embodiment” and the like are used and meant such that any of the embodiments described therein are to be read with a mind to combine each of the features of those embodiments and that the disclosure has to be treated in the same way as if the combination of the features of those embodiments would be spelled out in one embodiment. The same is true for any combination of embodiments and features of the appended claims and illustrated in the Examples, which are also intended to be combined with features from corresponding embodiments disclosed in the description, wherein only for the sake of consistency and conciseness the embodiments are characterized by dependencies while in fact each embodiment and combination of features, which could be construed due to the (multiple) dependencies must be seen to be literally disclosed and not considered as a selection among different choices. In this context, the person skilled in the art will appreciate that the embodiments and features disclosed in the Examples are intended to be generalized to equivalents having the same function as those exemplified therein.

### BRIEF DESCRIPTION OF THE DRAWINGS

FIG. 1 shows a schematic of Study Design including Aducanumab doses.

FIG. 2 shows the change from Baseline in Aβ PET Composite SUVR (reference region=cerebellum) by MMRM—¹⁸F-florbetapir Aβ PET Analysis Population—Study 2.

FIG. 3 shows the change from Baseline in Aβ PET Composite SUVR (reference region=cerebellum) by MMRM_¹⁸F-florbetapir Aβ PET Analysis Population—Study 1.

FIG. 4 shows CSF p-Tau change at 18 months in Study 1 and 2 using ANCOVA, CSF modified analysis population.

FIG. 5 shows CSF t-Tau change at 18 months in Study 1 and 2 using ANCOVA, CSF modified analysis population.

FIG. 6 shows the mean Aβ PET Composite SUVR time profiles for patients with ≥10 doses of 10/mg/kg at steady state

FIG. 7 shows the mean CDR-SB time profiles of those individuals from Study 1 and 2 who belong to the to the groups with ≥6 SS dosing intervals at 10 mg/kg, ≥8 SS dosing intervals at 10 mg/kg, and ≥10 SS dosing intervals at 10 mg/kg.

FIG. 8A contains scatter plots showing the correlation in Study 2 and Study 1 of cerebrospinal fluid (CSF) p-tau levels with cumulative aducanumab dose up to Week 78. Squares represent low dose aducanumab. Triangles represent high dose aducanumab (see FIG. 1).

FIG. 8B contains scatter plots showing the correlation in Study 2 and Study 1 of CSF total-tau levels with cumulative aducanumab dose up to Week 78. Squares represent low dose aducanumab. Triangles represent high dose aducanumab (see FIG. 1).

FIGS. 9A and 9B examine tau deposition in the medial temporal composite of the brain of study subjects. FIG. 9A is a graph showing adjusted mean change from baseline in tau positron emission tomography (PET) average standardized uptake value ratio (SUVR), assessed by ¹⁸F-MK-6240 in the tau PET study. Values based on an analysis of covariance (ANCOVA) model at Week 78, fitted with change from baseline as dependent variable, and with categorical treatment, baseline tau PET value and laboratory APOE e4 status (carrier and non-carrier) as independent variables. P Values: ***P<0.001 compared with placebo (nominal). Due to the early termination of the studies, all the post-baseline tau PET assessments were performed within a range of 9 to 20 months post-baseline in the placebo-controlled period. FIG. 9B is a scatter plot of change from baseline in medial temporal composite SUVR in correlation with cumulative dose by Week 78. SE, standard error.

FIGS. 10A, 10B, and 10C examine the cognitive progression and Amyloid PET biomarker data from one AD patient (Subject 218-110) treated with placebo during the Phase 1b Trial (Study 221-AD-103) and with aducanumab during the Long Term Extension (LTE). FIG. 10A is a graphical depiction of CDR-SB (dark line) and MMSE (gray line) data progression from initial patient screening through the Phase 1b (Placebo) and the LTE (Aducanumab). Screening data are shown to the left of the y-axis. The cognitive data points highlighted in red are those measurements that immediately preceded aducanumab administration and are consistent with mild-to-moderate dementia prior to enrollment in the LTE. FIG. 10B shows axial slice Amyoid PET (florbetapir) at baseline (ton row). Weeks 26 and 54 (rows 2 and 3) in the Placebo arm of the Phase 1b trial and at Weeks 110 and 166 (rows 4 and 5) of the LTE. These images demonstrate reduction in Amyloid PET standardized uptake value ratio (SUVR), indicative of Aβ plaque reduction, following administration of aducanumab. FIG. 10C is a graphical presentation of composite and regional SUVR values. Taking the graphs at 0 weeks, the lowest graph is striatal; the next is composite; the next is frontal, and the top most is occipital

SUVR. They demonstrate Amyloid plaque reduction in frontal cortex, occipital cortex and striatum.

FIGS. 11A-11F provide Aβ immunohistochemical stains (6E10 antibody) that demonstrate sparse residual Aβ plaques comprised predominantly of dense cores following aducanumab treatment. FIGS. 11A-11D show low- and high-power magnification images of frontal neocortex from an untreated HIGH AD neuropathology case of the Yale ADRC research cohort (11A, 11C) demonstrating frequent cortical Aβ plaques and amyloid angiopathy. Images from the LTE subject exhibit sparse cortical Aβ plaques predominantly comprised of dense cores surrounded by reactive microglia (11B, 11D). Dense cores surrounded by moth-eaten peripheral halos of non-compact Aβ were most prevalent in the occipital neocortex (inset 11D). Amyloid angiopathy was also identified. FIG. 11E shows heat maps generated from 6E10-immunostained sections of middle frontal (left column), mesiotemporal (middle column) and parastriate cortices (right column) demonstrating lower Aβ plaque immunoreactivity in our PRIME LTE Subject (lower row) compared to an untreated HIGH AD neuropathology control (upper row). FIG. 11F shows a graphical comparison of a very low density of temporal neocortical Aβ plaques in the

PRIME LTE Subject compared to a higher range of temporal neocortical Aβ plaque densities in a group of 9 untreated HIGH AD controls.

FIGS. 12A-12D illustrate microglia surrounding residual dense core AP plaques and demonstrating amoeboid reactive morphology. FIGS. 12A and 12B are low-power images of sections from an untreated HIGH AD case control (FIG. 12A) and the LTE Patient (FIG. 12B) reacted with duplex IBA1/6E10 immunohistochemical staining protocols. Highly-reactive, amoeboid microglial morphology is noted around residual plaques in the LTE Patient. FIG. 12C graphically depicts the quantitation of IBA1 immunoreactive processes within 5 microns of AP plaque and demonstrates increased plaque engagement by microglia in the LTE Patient compared to a cohort of untreated HIGH AD case controls. FIG. 12D depicts microglia (IBA1) surrounding residual dense core Aβ plaques showing reactive amoeboid morphology and cytoplasmic staining consistent with Aβ phagocytosis.

FIGS. 13A-13E are phosphorylated TAU (pTAU; 40E8) immunohistochemistry studies demonstrating sparse neocortical neuritic plaques (NPs) in the LTE subject. FIG. 13A shows sections of middle frontal neocortex from an untreated HIGH AD neuropathology control from the Yale ADRC research cohort (top row), an untreated HIGH AD neuropathology control from the Netherlands Brain Bank (NBB, middle row) and the LTE Subject (bottom row). Left column: low power images (original magnification 2.5×) show dense pTAU immunohistochemical reactivity in the HIGH AD sections from Yale and NBB compared to the LTE Subject. Middle column: medium power images of the regions identified by boxes in left column demonstrating frequent NPs (arrows) in HIGH AD sections from Yale and NBB but no NPs in the LTE Subject section. Right column: High power images demonstrating NPs (arrows), frequent NFTs and dense NTs in in HIGH AD sections from Yale and NBB. The section from the LTE Patient shows comparatively fewer NFTs and NTs. FIG. 13B shows a section of mesiotemporal lobe including hippocampus, parahippocampal gyms and occipitotemporal gyms from a HIGH AD neuropathology case from Yale (top row) and the PRIME LTE Subject (bottom row). Left column: low power images (original magnification 2.5×) show dense pTAU immunohistochemical reactivity in the occipitotemporal neocortex in the HIGH AD case from Yale compared to the LTE Subject. Reactivity in the parahippocampal gyms is more comparable in these 2 cases. Middle column: medium power images of the occipitotemporal neocortical regions identified by boxes in left column demonstrating frequent NPs (arrows) in the HIGH AD section from Yale but no NPs in the LTE Subject section. Right column: High power images demonstrating NPs (arrows), frequent NFTs and dense NTs in in HIGH AD sections from Yale and NBB. The section from the LTE Patient shows comparatively fewer NFTs and NTs. FIG. 13C shows a graphical comparison of a very low density of temporal neocortical pTau neuropathology in the PRIME LTE Subject compared to a higher range of temporal neocortical pTau neuropathology in a group of 9 untreated HIGH AD controls. FIG. 13D shows representative images of dual Aβ/pTAU immunoreactivity in NPs. Upper panel: NPs in HIGH AD patients demonstrate the usual pTAU-immunoreactive dystrophic neurites (NP Tau). Lower panel: few pTAU-immunoreactive dystrophic neurites around a residual dense-core amyloid [symbol] density of NP Tau neuropathology in the PRIME LTE Subject compared to a higher range of temporal neocortical NP Tau neuropathology in a group of 9 untreated HIGH AD controls.

FIG. 14A-14D show the mean change from baseline in the CDR-SB, MMSE, ADAS-Cog13, and ADCS-ADL-MCI scores over 78 weeks. Longitudinal change from baseline in clinical measures in the ITT population is presented here. FIG. 14A shows the mean change from baseline in the CDR-SB score; scores range from 0 to 18, with higher scores indicating greater impairment. FIG. 14B shows the mean change from baseline in the MMSE score; scores range from 0 to 30, with lower scores indicating greater impairment. FIG. 14C shows the mean change from baseline in the ADAS-Cog 13 score; scores range from 0 to 85, with higher scores indicating greater impairment. FIG. 14D shows the mean change from baseline in the ADCS-ADL-MCI score; scores range from 0 to 53, with lower scores indicating greater impairment. Values at each time point were based on an MMRM model, with change from baseline in CDR-SB, MMSE, ADAS-Cog 13, or ADCS-ADL-MCI score as the dependent variable and with fixed effects of treatment group, categorical visit, treatment-by-visit interaction, baseline measure, baseline measure—by-visit interaction, baseline MMSE score (same as baseline score in the MMSE model), Alzheimer's disease symptomatic medication use at baseline, region, and laboratory ApoE ϵ4 status. P values: †P<0.1 and >0.05, *P<0.05, **P<0.01, ***P<0.001. Error bars denote standard error. ADAS-Cog13, Alzheimer's Disease Assessment Scale, 13-item; ADCS-ADL-MCI, Alzheimer's Disease Cooperative Study—Activities of Daily Living Inventory, mild cognitive impairment version; adu, aducanumab; ApoE, apolipoprotein E; CDR- SB, Clinical Dementia Rating Scale—Sum of Boxes; MMRM, mixed model for repeated measures; MMSE, Mini-Mental State Examination; SE, standard error.

FIG. 15 shows longitudinal change from baseline in amyloid PET average standardized uptake value ratio (SUVR), assessed by ¹⁸F-florbetapir in the amyloid PET substudy. Composite SUVR was computed from the frontal, parietal, lateral temporal and sensorimotor, anterior, and posterior cingulate cortices and normalized using the cerebellum as the reference region. Change from baseline in amyloid PET SUVR was analyzed using an MMRM model with fixed effects of treatment, categorical visit, treatment-by-visit interaction, baseline SUVR, baseline SUVR—by-visit interaction, baseline MMSE, laboratory ApoE E4 status (carrier and noncarrier), and baseline age. Placebo (diamond) value denotes the adjusted mean change from baseline at Week 78. Low-dose (square) and high-dose (triangle) aducanumab values denote the difference from placebo at Week 78. ***P<0.001. Error bars denote SE. adu, aducanumab; ApoE, apolipoprotein E; MMRM, mixed model for repeated measure; MMSE, Mini Mental State Examination; PET, positron emission tomography; SE, standard error.

FIG. 16 shows CSF Aβ1-42 at Week 78. The figures shows adjusted mean change from baseline in CSF Aβ1-42 values in the CSF substudy. Values were based on an ANCOVA model at Week 78, fitted with change from baseline as the dependent variable, and with treatment, baseline CSF Aβ1-42 value, baseline age, and laboratory ApoE ϵ4 status (carrier and noncarrier) as the independent variables. P values: ***P<0.001. ANCOVA, analysis of covariance; ApoE, apolipoprotein E; CSF, cerebrospinal fluid; SE, standard error. FIG. 17 shows CSF p-tau and t-tau at Week 78. Adjusted mean change from baseline in

CSF levels of p-tau and t-tau in the CSF substudy. Values were based on an ANCOVA model at Week 78, fitted with change from baseline as the dependent variable, and with treatment, baseline biomarker value, baseline age, and laboratory ApoE ϵ4 status (carrier and noncarrier) as the independent variables. P values: *P<0.05, **P<0.01, and ***P<0.001. ANCOVA, analysis of covariance; ApoE, apolipoprotein E; CSF, cerebrospinal fluid; p-tau, phosphorylated tau 181; SE, standard error; t-tau, total tau.

FIG. 18 shows tau deposition in the medial temporal composite. Adjusted mean change from baseline in tau PET average SUVR assessed by ¹⁸F-MK-6240 in the tau PET substudy. Values based on an ANCOVA model at Week 78, fitted with change from baseline as the dependent variable, and with categorical treatment, baseline tau PET value, and laboratory ApoE ϵ4 status (carrier and noncarrier) as independent variables. P values: *P<0.05, **P<0.01, and ***P<0.001. ANCOVA, analysis of covariance; PET, positron emission tomography; SE, standard error; SUVR, standardized uptake value ratio.

### DETAILED DESCRIPTION

### Alzheimer's Disease

Alzheimer's disease, abbreviated herein as AD, is a dementia that is primarily identified by clinical diagnosis and established by markers of the disease.

AD is a continuum having certain operationally defined stages of disease progression. AD pathology begins prior to the onset of clinical symptoms. For example, amyloid plaques, one marker of AD pathology. form 10-29 vear prior to the onset of AD dementia. The currently recognized stages of AD include preclinical, prodromal, mild, moderate, and severe. These stages may be further divided into subcategories based on the severity of symptoms and measures of AD progression.

Because AD does not occur in discrete stages, those skilled in the art will recognize that the differences between patient groups may not be distinct in a particular clinical setting.

Nevertheless, the clinical disease stage can be characterized by measures, and changes in these measures over time, such as Aβ accumulation (CSF/PET), synaptic dysfunction (FDG-PET/fMRI), tau-mediated neuronal injury (CSF), brain structure (volumetric MRI), cognition, and clinical function. (Jack C R, et al. Hypothetical model of dynamic biomarkers of the Alzheimer's pathological cascade. Lancet Neurol., 2010; 9(1):119-28).

Current core clinical criteria for all dementia, referred to as the NINCDS-ADRDA criteria (McKhann G M, V. diagnosis of dementia due to Alzheimer's disease: Recommendations from the National Inst. on Aging-Alzheimer's Association workgroups on diagnostic guidelines for Alzheimer's disease. Alzheimer's & Dementia, 7 (2011) 263-269), are known in the art and can be employed in practicing this invention. They include cognitive or behavioral impairment involving impaired ability to acquire and remember new information, impaired reasoning and handling of complex tasks, impaired visuospatial abilities, impaired language functions (speaking, reading, writing), and changes in personality, behavior, or comportment. Alzheimer's disease is currently diagnosed using the core criteria and is typically characterized by symptoms which have a gradual onset over months to years, not sudden over hours or days (insidious onset). There is usually a clear-cut history of worsening of cognition by report or observation in Alzheimer's disease subjects.

Other diagnostic classification systems have evolved as new information on AD has become available. These systems include the International Working Group (IWG) new research criteria for diagnosis of AD (Dubois B et al., Lancet Neurol., 2007; 6(8):734-736), IWG research criteria, (Dubois et al., Lancet Neurol., 2010;9(11):1118-27), NIA/AA Criteria (Jack C R et al. Alzheimer's Dement., 2011;7(3):257-62), and DSM-5 criteria (American Psychiatric Association, DSM-5, 2013). These classification systems can also be employed in diagnosing AD subjects for treatment according to the methods of this disclosure.

### Patients

The term “patient” is meant to include any human subject for whom diagnosis, prognosis, prevention, or therapy for Alzheimer's disease is desired, and includes a human subject in need of treatment. Those in need of treatment include those already with AD, as well as those prone to have AD, or those in which the manifestation of AD is to be prevented. Typical patients will be men or women aged 40 to 90 (e.g., 45 to 90, 50 to 90, 55 to 90, 60 to 90). In one embodiment, the disclosure provides a method of treating a patient with AD (including, without limitation, patients with preclinical, prodromal, mild, moderate, or severe AD). In certain instances, the disclosure provides a method of treating a patient with prodromal Alzheimer's disease. In some instances, the disclosure provides a method of treating a patient with early Alzheimer's disease. In some instances, the disclosure provides a method of treating a patient to reduce clinical decline in Alzheimer's disease. In some instances, the disclosure provides a method of treating a patient with mild cognitive impairment due to Alzheimer's disease. In other instances, the disclosure provides a method of treating a patient with mild Alzheimer's disease dementia. In a further embodiment, the patient has amyloid pathology confirmed, e.g., by positron emission tomography (PET) imaging. In some cases, amyloid β pathology is confirmed by [¹⁸F]-florbetapir PET imaging. In some cases, amyloid β pathology is confirmed by ['^{R}F]- flutemetomol PET imaging. In some cases, amyloid β pathology is confirmed by [¹⁸F]-florbetaben PET imaging. In some cases, amyloid pathology is confirmed by CSF amyloid analysis. In some cases, amyloid β pathology is confirmed by blood amyloid β analysis. In some cases, amyloid β pathology is confirmed by Congo red staining and birefringence under polarized microscopy. In some cases, amyloid β pathology is confirmed by immunohistochemistry (IHC), electron microscopy, or mass spectrometry. In some cases, amyloid β pathology is confirmed by any method to assess levels of amyloid β.

In certain instances, the patient to be treated has an MMSE score between 24-30 (inclusive). In some instances, the patient to be treated has a CDR global score of 0.5. In some instances, the patient to be treated has a RBANS score of less than or equal to 85 (based upon Delayed Memory Index score). In some instances, the patient to be treated has at least 6 years of work experience. In some cases, the patient to be treated has an MMSE score between 24-30 (inclusive); a CDR global score of 0.5; and a RBANS score of less than or equal to 85 (based upon Delayed Memory Index score). In certain instances, the patient is an ApoE4 carrier (ApoE4 positive). In certain instances, the patient is an ApoE4 non-carrier (ApoE4 negative).

AD patients in need of treatment range from subjects with amyloid pathology and early neuronal degeneration to subjects with widespread neurodegeneration and irreversible neuronal loss with progressive cognitive and functional impairment to subjects with dementia.

Patients with preclinical AD can be identified by asymptomatic stages with or without memory complaints and emerging episodic memory and executive function deficits. This stage is typically characterized by the appearance of in vivo molecular biomarkers of AD and the absence clinical symptoms.

Prodromal AD patients are pre-dementia stage characterized predominantly by cognitive deficits and emerging functional impairment with disease progression. Prodromal AD patients typically have mini-mental state examination (MMSE) scores between 24-30 (inclusive), a spontaneous memory complaint, objective memory loss defined as a free recall score of <27 on the Free and Cued Selective Reminding Test (FCSRT), a global Clinical Dementia Rating (CDR) score of 0.5, absence of significant levels of impairment in other cognitive domains, and essentially preserved activities of daily living, and an absence of dementia.

Patients with mild AD typically have MMSE scores between 20-26 (inclusive), a global CDR of 0.5 or 1.0, and meet the National Institute on Aging-Alzheimer's Association core clinical criteria for probable AD (see Section 22).

Basing AD diagnosis on clinical symptoms, mild stage AD patients will exhibit conspicuous behavior at work, forgetfulness, mood swings, and attention disturbances. Moderate stage AD patients will exhibit cognitive deficits, restricted everyday activities, orientation disturbance, apraxia, agnosia, aphasia, and behavioral abnormalities. Severe stage AD patients are characterized by loss of independence, decay of memory and speech, and incontinence,

In certain embodiments, treatment is of earlier-stage patients who are amyloid positive as assessed by [¹⁸F]-florbetapir PET scans. In certain embodiments, treatment is of earlier-stage patients who are amyloid positive as assessed by ¹⁸F-flutemetomol PET scans. In certain embodiments, treatment is of earlier-stage patients who are amyloid positive as assessed by 18F-florbetaben PET scans. In certain instances, the human subject is confirmed to have a brain amyloid beta Pathology prior to the initiation of treatment. Thg patient may be asymptomatic for, or exhibit only transient symptoms of, headache, confusion, gait difficulties, or visual disturbances. The patient may or may not be an ApoE4 carrier as determined by ApoE genotyping.

In other embodiments, treatment is of patients having any medical or neurological condition (other than AD) that might be a contributing cause of the subject's cognitive impairment, such as stroke or other cerebrovascular condition, other neurodegenerative disease, a history of clinically significant psychiatric illness, acute or sub-acute micro- or macro hemorrhage, prior macrohemorrhage, or superficial siderosis. These patients can be treated following screening and selection by a qualified clinician.

### Anti-Beta Amyloid Antibody

Antibody BIIB037, also known as aducanumab, is a biologic treatment for Alzheimer's disease. It is an anti-AB antibody that recognizes aggregated forms of AB, including plaques. BIIB037 contains a human kappa light chain. BIIB037 consists of 2 heavy and 2 human kappa light chains connected by inter-chain disulfide bonds. By “BIIB037” or “aducanumab” is meant an anti-Aβ antibody comprising the amino acid sequences set forth in SEQ ID NOs: 10 and 11.

In vitro characterization studies have established that antibody BIIB037 recognizes a conformational epitope present in Aβ aggregates, the accumulation of which is believed to underlie the development and progression of AD. In vivo pharmacology studies indicate that a murine IgG2a chimeric version of the antibody (ch 12F6A) with similar properties significantly reduces amyloid plaque burden in the brains of aged Tg2576 mice, a mouse model of AD. The reduction in parenchymal amyloid was not accompanied by a change in vascular amyloid, as has been reported for certain anti-Aβ antibodies (Wilcock O M, Colton C A. Immunotherapy, vascular pathology, and microhemorrhages in transgenic mice. CNS & Neurological Disorders Drug Targets, 2009 Mar.;8(1):50-64).

The VH and VL of antibody BIIB037 have amino acid sequences that are identical to the amino acid sequence of the VH and VL of antibody NI-101.12F6A described in U.S. Pat. No. 8,906,367 (see, Tables 2-4; incorporated by reference in its entirety herein). Specifically, antibody BII3037 has an antigen binding domain comprising VH and VL variable regions depicted in Table A (VH) and Table B (VL). corresponding complementarity determining regions (CDRs) depicted in Table C, and heavy and light chains depicted in Table D (H) and Table E (L).

TABLE A
Amino acid sequences of the V_{H} region of anti-Aβ
antibody BIIB037 (VH CDRs (Kabat definition)
underlined).
Variable heavy chain sequence

QVQLVESGGG VVQPGRSLRL SCAASGFAFS SYGMHWVRQA
PGKGLEWVAV IWFDGTKKYY TDSVKGRFTI SRDNSKNTLY
LQMNTLRAED TAVYYCARDR GIGARRGPYY MDVWGKGTTV
TVSS (SEQ ID NO: 1)

TABLE B
Amino acid sequences of the V_{L} region of anti-Aβ
antibody BIIB037 (VL CDRs (Kabat definition)
underlined).
Variable light chain sequence (kappa or lambda)

DIQMTQSPSS LSASVGDRVT ITCRASQSIS SYLNWYQQKP
GKAPKLLIYA ASSLQSGVPS RFSGSGSGTD FTLTISSLQP
EDFATYYCQQ SYSTPLTFGG GTKVEIKR (SEQ ID NO: 2)

TABLE C
Denomination of CDR protein sequences in Kabat Nomenclature of V_{H}
and V_{L} regions of anti-Aβ antibody BIIB037.

| CDR | Variable heavy chain | Variable light chain |
|---|---|---|
| CDR | SYGMH (SEQ ID NO: 3) | RASQSISSYLN (SEQ ID NO: 6) |
| CDR2 | VIWFDGTKKYYTDSVKG (SEQ ID NO: 4) | AASSLQS (SEQ ID NO: 7) |
| CDR3 | DRGIGARRGPYYMDV (SEQ ID NO: 5) | QQSYSTPLT (SEQ ID NO: 8) |

The amino acid sequence of the mature heavy chain of BIIB037 is provided in Table D below.

TABLE D:
Amino acid sequences of the heavy chain of anti-Aβ antibody BIIB037
(heavy chain CDRs (Kabat definition) underlined).
Heavy chain sequence

QVQLVESGGG VVQPGRSLRL SCAASGFAFS SYGMHWVRQA PGKGLEWVAV
IWFDGTKKYY TDSVKGRFTI SRDNSKNTLY LQMNTLRAED TAVYYCARDR
GIGARRGPYY MDVWGKGTTV TVSSASTKGP SVFPLAPSSK STSGGTAALG
CLVKDYFPEP VTVSWNSGAT TSGVHTFRAY LQSSGLYSLS SVVTVPSSSL
GTQTYICNVN HKPSNTKVDK RVEPKSCDKT HTCPPCPAPE LLGGPSVFLE
PPKPKDTLMI SRTPEVTCVV VDVSHEDPEV KFNWYVDGVE VHNAKTKPRE
EQYNSTYRVV SVLTVLHQDW LNGKEYKCKV SNKALPAPIE KTISKAKGQP
REPQVYTLPP SREEMTKNQV SLTCLVKGFY PSDIAVEWES NGQPENNYKT
TPPVLDSDGS FFLYSKLTVD KSRWQQGNVF SCSVMHEALH NHYTQKSLSL
SPG (SEQ ID NO: 10)

The amino acid sequence of the mature light chain of B1113037 is provided in Table E below.

TABLE E
Amino acid sequences of the light chain of anti-Aβ antibody
BIIB037 (light chain CDRs (Kabat definition) underlined).
Light chain sequence

DIQMTQSPSS LSASVGDRVT ITCRASQSIS SYLNWYQQKP GKAPKLLIYA
ASSLQSGVPS RFSGSGSGTD FTLTISSLQP EDFATYYCQQ SYSTPLTFGG
GTKVEIKRTV AAPSVFIFPP SDEQLKSGTA SVVCLLNNFY PREAKVQWKV
DNALQSGNSQ ESVTEQDSKD STYSLSSTLT LSKADYEKHK VYACEVTHQG
LSSPVTKSFN RGEC (SEQ ID NO: 11)

In addition to antibody BIIB037, this disclosure contemplates the use of the other anti-beta-amyloid antibodies, such as antibodies comprising either the VH region comprising or consisting of SEQ ID NO:1 or the VL region comprising or consisting of SEQ ID NO:2, or antibodies comprising the VH region comprising or consisting of SEQ ID NO:1 and the VL region comprising or consisting of SEQ ID NO:2, wherein the VH and/or VL regions have one or more substitutions, deletions, and/or insertions. In some embodiments, these VH and VL regions may have up to 25, up to 20, up to 15, up to 10, up to 5, or 1, 2, 3, 4, 5, 6, 7, 8, 9, 10, 11, 12, 13, 14, 15, 16, 17, 18, 19, or 20 amino acid substitutions and still bind beta-amyloid. In specific embodiments, these amino acid substitutions occur only in the framework region. In some embodiments, the amino acid substitution(s) is/are conservative amino acid substitutions. In certain embodiments, the VH and VL regions may include 1 to 5 (1, 2, 3, 4, 5) amino acid deletions and/or additions and still bind beta-amyloid. In certain embodiments, these deletions and/or additions are made at the N- and/or C-terminus of the VH and/or VL regions. In one embodiment, one amino acid is deleted and/or added at the N and/or C-terminus of the VH region. In one embodiment, one amino acid is deleted and/or added at the N and/or C-terminus of the VL region.

Other antibodies contemplated for use in the disclosure include antibodies comprising the variable heavy chain (VH) CDRs and the variable light chain (VL) CDRs in Table C. Thus, the anti-beta amyloid antibodies comprise the CDRs comprising or consisting of the amino acid sequences of SEQ ID NOs.: 3-8. In one embodiment, the anti-beta amyloid antibodies comprise the CDRs comprising or consisting of the amino acid sequences of SEQ ID NOs.: 4-8 and include as VH CDR1 an amino acid sequence comprising or consisting of GFAFSSYGMH (SEQ ID NO:9). In some instances, the disclosure encompasses anti-beta-amyloid antibodies comprising the VH and VL CDRs of BIIB037 based on any CDR definition (e.g., Kabat, Chothia, enhanced Chothia, AbM, or contact definition). See, e.g., http://www.bioinf.org.uk/abs/index.html. In one embodiment, the disclosure encompasses anti-beta-amyloid antibodies comprising the VH and VL CDRs of BIIB037 based on the Chothia definition. In one embodiment, the disclosure encompasses anti-beta-amyloid antibodies comprising the VH and VL CDRs of BIIB037 based on the enhanced Chothia definition. In another embodiment, the disclosure encompasses anti-beta-amyloid antibodies comprising the VH and VL CDRs of BIIB037 based on the AbM definition. In yet another embodiment, the disclosure encompasses anti-beta-amyloid antibodies comprising the VH and VL CDRs of BIIB037 based on the contact definition.

Antibody BIIB037 and other antibodies employed in the invention can be prepared using known methods. In some embodiments, the antibody is expressed in a Chinese hamster ovary (CHO) cell line.

The maximum tolerated amount of the anti-Aβ antibody is that quantity of the antibody which will produce a clinically significant response in the treatment of Alzheimer's disease consistent with safety. A principal safety concern in treating patients according to the method of the invention is the occurrence of ARIA, especially ARIA-E or ARIA-H. The methods of the invention make it possible to employ higher doses of antibody BIIB037 for the treatment of patients for AD than was feasible using previously known protocols.

It will be understood that dose adjustments can be implemented during the treatment protocol. For example, for reasons of safety or efficacy, doses can be increased so that the effects of the anti-Aβ antibody on AD can be enhanced or doses can be decreased so that the ARIA rate and severity can be mitigated. If a dose is missed, the patient should preferably resume dosing by receiving the missed dose and continuing thereafter according to the described regimen.

In certain embodiments, the anti-Aβ antibody is administered to the patient by intravenous infusion following dilution into saline. When using this mode of administration, each infusion step in the titration regime of the invention will typically take about 1 hour.

The dose ranges and other numerical values herein include a quantity that has the same effect as the numerically stated amount as indicated by treatment of Alzheimer's disease in the patient and a reduction in the incidence or susceptibility of the patient to ARIA when compared to an individual not treated by the method of the invention. At the very least, each numerical parameter should be construed in light of the number of significant digits, applying ordinary rounding techniques. In addition, any numerical value inherently contains certain errors from the standard deviation of its measurement and such values are within the scope of the invention.

### Treatment

As used herein, the terms “treat” or “treatment” generally mean obtaining a desired pharmacological and/or physiological effect in the subject being administered the anti-beta amyloid antibody. Hence, the term “treatment” as used herein includes: (a) inhibiting AD, e.g. arresting its development; (b) relieving AD, e.g. causing regression of AD; or (c) prolonging survival as compared to expected survival if not receiving treatment.

In one embodiment, the treatment is therapeutic. In another embodiment, treatment has a disease modifying effect. This means that the treatment slows or delays the underling pathological or pathophysiological disease processes and there is an improvement in clinical signs and symptoms of AD relative to placebo.

In a further embodiment, treatment results in symptomatic improvement. This may consist of enhanced cognition, more autonomy, and/or improvement in neuropsychiatric and behavioral dysfunction, even if for only a limited duration.

In another embodiment, the disclosure relates to methods for delaying clinical decline or progression of disease, or relief of symptoms. Delaying clinical decline or disease progression directly impacts the patient and care-givers. It delays disability, maintains independence, and allows the patient to live a normal life for a longer period of time. Relief of symptoms to the best degree possible can incrementally improve cognition, function, and behavioral symptoms, as well as mood.

This disclosure features a titration regimen (sequential administration of increasing doses of the anti-beta amyloid antibody) to treat Alzheimer's disease. In some instances, the Alzheimer's disease is mild Alzheimer's disease, early Alzheimer's disease, prodromal Alzheimer's disease, mild Alzheimer's disease dementia, or mild cognitive impairment due to Alzheimer's disease.

In one method of treatment of Alzheimer's disease, the anti-beta amyloid antibody is administered to a human patient in increasing amounts over a period of time. This procedure of sequentially administering the antibody to the patient is referred to herein as “titration” because it involves administering a standardized pharmaceutical of known concentrations in carefully measured amounts until completion of the procedure.

One of the advantages of the titration regime of the invention is that it makes it possible to administer higher doses of the monoclonal antibody to AD patients without incurring the same extent of ARIA observed with a standard dose regimen. In certain embodiments, the higher dose comprises a dose or doses of the anti-Aβ antibody of 10 mg/kg of the body weight of the subject. Without intending to be limited to any particular mechanism, it is believed that titration results in lower initial amyloid removal and slower removal during the overall treatment.

Titration of the anti-Aβ antibody (e.g., BIIB037) is carried out in multiple doses. For example, two doses of the antibody can be administered to the patient in an amount per dose that is less than the minimum therapeutic amount, followed by 4 doses of the antibody in an amount per dose that is about equal to the minimum therapeutic amount. This regime can then be followed by multiple doses in an amount per dose that is more than the minimum therapeutic amount, but less than the maximum tolerated amount until there is an acceptable change in AD in the patient. For example, doses can be administered approximately 4 weeks apart over approximately 52 weeks (a total of 14 doses). Progress can be monitored by periodic assessment.

In some instances, the disclosure features a method for reducing tau or treating Alzheimer's disease by reducing Abeta and/or tau in a human patient in need thereof, the method comprising sequentially administering multiple doses of an anti-Aβ antibody (e.g., BIIB037) in increasing amounts over a period of time to the human patient, wherein multiple doses of 1 mg antibody/kg of body weight of the human patient are administered to the human patient at intervals of about 4 weeks: multiple doses of 3 g antibody/kg of the human patient are administered to the human patient at intervals of about 4 weeks; multiple doses of 6 mg antibody/kg of body weight of the human patient are administered to the human patient at intervals of about 4 weeks; and multiple doses of 10 mg antibody/kg of body weight of the human patient are administered to the human patient at intervals of about 4 weeks. Multiple doses means at least two (e.g., 2, 3, 4, 5, 6, 7, 8, 9, 10, 11, 12, 123, 14, 15, 16, 17, 18, 19, 20, 21, 22, 23, 24, 25, 26, 27, 28, 29, 30) doses.

One protocol according to the disclosure, designated Protocol A, comprises:

(A) administering the anti-beta amyloid antibody to the patient in an amount of 1 mg/kg of body weight of the patient;

(B) 4 weeks after step (A), administering the anti-beta amyloid antibody to the patient in an amount of 1 mg/kg of body weight of the patient;

(C) 4 weeks after step (B), administering the anti-beta amyloid antibody to the patient in an amount of 3 mg/kg of body weight of the patient;

(D) 4 weeks after step (C), administering the anti-beta amyloid antibody to the patient in an amount of 3 mg/kg of body weight of the patient;

(E) 4 weeks after step (D), administering the anti-beta amyloid antibody to the patient in an amount of 6 mg/kg of body weight of the patient;

(F) 4 weeks after step (E), administering the anti-beta amyloid antibody to the patient in an amount of 6 mg/kg of body weight of the patient; and

(G) in consecutive intervals of 4 weeks after step (F), administering the anti-beta amyloid antibody to the patient in an amount of 10 mg/kg of body weight of the patient.

In other words, Protocol A comprises administering a first dose of anti-beta amyloid antibody to the patient in an amount of 1 mg/kg of body weight of the patient, followed by a second dose in an amount of 1 mg/kg of body weight four weeks after the first dose. In four week intervals after the second dose, antibody doses 3 and 4 are administered to the patient in an amount of 3 mg/kg of body weight. In four week intervals after administration of dose 4, doses 5 and 6 of the antibody are administered to the patient in an amount of 6 mg/kg of body weight. And then, four weeks after administration of dose 6, antibody dose 7 is administered to the patient in an amount of 10 mg/kg of body weight. In some instances, after dose 7 of Protocol A, 5, 6, 7, 8, 9, or 10 doses of the anti-beta amyloid antibody in an amount of 10 mg/kg are administered to thg patient. In certain instances, at least 10, at least 11, at least 12, at least 13, or at least 14 doses of the anti-beta amyloid antibody in an amount of 10 mg/kg of body weight of the subject are administered to the patient. In certain instances, at least 15, at least 16, at least 17, at least 18, at least 19, or at least 20 doses of the anti-beta amyloid antibody in an amount of 10 mg/kg of body weight of the subject are administered to the patient. In certain instances, 15 to 16, 15 to 17, 15 to 18, 15 to 19, 15 to 20, 15 to 21, 15 to 22, 15 to 23, 15 to 24, or 15 to 25 doses of the anti-beta amyloid antibody in an amount of 10 mg/kg of body weight of the subject are administered to the patient. In certain instances, the doses mentioned above are administered in consecutive intervals of 4 weeks. In certain instances, the doses mentioned above are administered to the patient intravenously.

In some instances, after dose 7 of Protocol A, at least 10 doses of the anti-beta amyloid antibody in an amount of 10 mg/kg of body weight of the subject are administered (e.g., intravenously) to the patient in uninterrupted 4 week intervals.

Another protocol according to the disclosure, designated Protocol B, comprises:

(a) administering the anti-beta-amyloid antibody to the subject in an amount of 1 mg/kg of body weight of the subject;

(b) 4 weeks after step (a), administering the anti-beta-amyloid antibody to the subject in an amount of 3 mg/kg of body weight of the subject;

(c) 4 weeks after step (b), administering the anti-beta-amyloid antibody to the subject in an amount of 6 mg/kg of body weight of the subject; and

(d) in consecutive intervals of 4 weeks after step (c), administering at least 10 doses of the anti-beta-amyloid antibody in an amount of 10 mg/kg of body weight of the subject.

In some instances, after step (d) of Protocol B, additional doses of the anti-beta amyloid antibody in an amount of 10 mg/kg of body weight are administered to the patient. In certain instances, at least 11, at least 12, at least 13, at least 14, at least 15, at least 16, at least 17, at least 18, at least 19, or at least 20 doses of the anti-beta amyloid antibody in an amount of 10 mg/kg of body weight of the subject are administered to the patient. In certain instances, at least 21, at least 22, at least 23, at least 24, at least 24, or at least 25 doses of the anti-beta amyloid antibody in an amount of 10 mg/kg of body weight of the subject are administered to the patient. In certain instances, 11 to 12, 11 to 13, 11 to 14, 11 to 15, 11 to 16, 11 to 17, 11 to 18, 11 to 19, 11 to 20. or 11 to 25 doses of the anti-beta amyloid antibody in an amount of 10 mg/kg of body weight of the subject are administered to the patient. In certain instances, the additional doses mentioned above are administered in consecutive intervals of 4 weeks. In certain instances, the doses mentioned above are administered to the patient intravenously.

In certain instances, when the patient develops an Amyloid Related Imaging Abnormality (ARIA)—e.g., ARIA-E, during the course of treatment is suspended until the ARIA resolves. In some instances, the treatment is suspended for 1 to 15 (1, 2, 3, 4, 5, 6, 7, 8, 9, 10, 11, 12, 13, 14, 15) weeks for the ARIA to resolve and then restarted. In certain cases, if the subject develops ARIA-E and/or ARIA-H accompanied by serious clinical symptoms, or ARIA-H with greater than or equal to 10 microhemorrhages and/or greater than or equal to two focal areas of superficial siderosis, or any new incident macrohemorrhage, treatment is permanently discontinued.

In certain instances, when the patient develops an Amyloid Related Imaging Abnormality (ARIA) during the course of treatment under Protocol A or B, the patient continues to be administered the doses described above without reduction of the dosage. In some instances, the dosage may be administered after the ARIA resolves.

If for some reason there is an interruption in the treatment (e.g., missed visits to the doctor or doctor recommendations due to ARIA or other side effects), the patient upon resumption of the treatment should continue at the same or higher dose. For example if the patient has already received two 3 mg/kg doses of the anti-beta amyloid antibody before interruption, upon resumption of treatment, the patient should be administered the 6 mg/kg dose.

If the patient has already received two 6 mg/kg doses of the anti-beta amyloid antibody before interruption, upon resumption of treatment, the patient should be administered the 10 mg/kg dose. If the patient has already received two 10 mg/kg doses of the anti-beta amyloid antibody before interruption, upon resumption of treatment, the patient should be administered a 10 mg/kg dose and continue to be administered the 10 mg/kg dose for as long as possible.

The disclosure also features a method for treating mild Alzheimer's disease, early Alzheimer's disease, prodromal Alzheimer's disease, mild Alzheimer's disease dementia, or mild cognitive impairment due to Alzheimer's disease in a human subject in need thereof. The method involves administering to the human subject multiple doses of an anti-beta-amyloid antibody, wherein the method comprises administering in consecutive intervals of 4 weeks at least 6 doses of the antibody, wherein each dose is in an amount of 10 mg/kg of body weight of the subject. In some instances, the method comprises administering in consecutive intervals of 4 weeks at least 7 doses of the antibody, wherein each dose is in an amount of 10 mg/kg of body weight of the subject. In some instances, the method comprises administering in consecutive intervals of 4 weeks at least 8 doses of the antibody, wherein each dose is in an amount of 10 mg/kg of body weight of the subject. In some instances, the method comprises administering in consecutive intervals of 4 weeks at least 9 doses of the antibody, wherein each dose is in an amount of 10 mg/kg of body weight of the subject. In some instances, the method comprises administering in consecutive intervals of 4 weeks at least 10 doses of the antibody, wherein each dose is in an amount of 10 mg/kg of body weight of the subject. In some instances, the method comprises administering in consecutive intervals of 4 weeks at least 11 doses of the antibody, wherein each dose is in an amount of 10 mg/kg of body weight of the subject. In some instances, the method comprises administering in consecutive intervals of 4 weeks at least 12 doses of the antibody, wherein each dose is in an amount of 10 mg/kg of body weight of the subject. In some instances, the method comprises administering in consecutive intervals of 4 weeks at least 13 doses of the antibody, wherein each dose is in an amount of 10 mg/kg of body weight of the subject. In some instances, the method comprises administering in consecutive intervals of 4 weeks at least 14 doses of the antibody, wherein each dose is in an amount of 10 mg/kg of body weight of the subject. In some instances, the method comprises administering in consecutive intervals of 4 weeks at least 15 doses of the antibody, wherein each dose is in an amount of 10 mg/kg of body weight of the subject. In certain instances, all of the doses specified are administered without interruption even if the human subject develops an ARIA during the course of treatment. In certain instances, even if the doses are interrupted due to ARIA or other side effects, the treatment is continued at the same or higher dose of the antibody. If the patient was at the highest dose of Protocol A (10 mg/kg), upon resuming treatment after an interruption, the patient is to continue to be administered a 10 mg/kg dose of the antibody.

In some instances, the anti-beta amyloid antibody of the protocols and methods above comprises a VH and VL comprising the six CDRs of BIIB037. In certain instances, the anti-beta amyloid antibody comprises the VH and VL of BIIB037. In other instances, the anti-beta amyloid antibody comprises the heavy and light chains of BIIB037. In some instances, the anti-beta-amyloid antibody comprises a heavy chain variable region (VH) and a light chain variable region (VL). wherein the VII comprises a complementarity determining region (VHCDR1) with the amino acid sequence of SEQ ID NO:3, a VHCDR2 with the amino acid sequence of SEQ ID NO:4, and a VHCDR3 with the amino acid sequence of SEQ ID NO:5, and wherein the VL comprises a VLCDRI with the amino acid sequence of SEQ ID NO:6, a VLCDR2 with the amino acid sequence of SEQ ID NO:7, and a VLCDR3 with the amino acid sequence of SEQ ID NO:8. In some instances, the anti-beta amyloid antibody comprises a VH comprising or consisting of SEQ ID NO:1; and a VL comprising or consisting of SEQ ID NO:2. In some cases, the anti-beta-amyloid antibody comprises a heavy chain and a light chain, wherein: the heavy chain comprises or consists of SEQ ID NO:10; and the light chain comprises or consists of SEQ ID NO:11.

### EXAMPLES

### Example 1: Overview of Phase 3 Studies

The efficacy and safety of aducanumab in subjects with mild cognitive impairment (MCI) due to Alzheimer's disease or mild Alzheimer's disease dementia was evaluated in 2 identically designed Phase 3 studies, Study 1 and Study 2. The studies were designed based on an understanding of the mechanism of action of aducanumab, the outcomes of earlier studies, and the current understanding of the disease process and the underlying pathology. Table 1 provides an overview of the study design.

TABLE I

| | Overview of Phase 3 Study Design |
|---|---|
| Population | 1. MCI due to AD (~75-80%) + mild AD (~25-20%) |
| | 2. MMSE 24-30, CDR global score 0.5, Repeatable Battery |
| | for the Assessment of Neuropsychological Status (RBANS) |
| | delayed memory index ≤85 |
| | 3. Amyloid positive (by Aβ PET) |
| | 4. 50-85 years of age |
| | 5. ≥6 years of education or work experience |
| | 6. Use of stable Alzheimer's disease symptomatic |
| | medication on entry allowed |
| Study | 18-month placebo-controlled period + up to 5 years long- |
| duration | term extension (LTE) period |
| Sample | ~N = 1605 per study (n = 535/ group) |
| size | Subjects randomized to placebo: low dose: high dose: in a |
| | 1:1:1 ratio |
| | Randomization stratified by site and apolipoprotein (ApoE) |
| | ε4 status (carrier or non-carrier) |
| Doses | Two aducamimab dose levels (low and high). Differential |
| | dosing based on ApoE ε4 status. |

These Phase 3 studies recruited earlier-stage patients who were Aβ positive as assessed by Aβ PET scans (by visual read) and who fulfilled clinical criteria for either MCI due to Alzheimer's disease or mild Alzheimer's disease dementia (as defined by NIA-AA criteria). Enrollment was monitored such that approximately 80% of the Phase 3 study populations would include subjects with a baseline clinical stage of MCI due to Alzheimer's disease (per the investigator's clinical assessment). Subjects were also required to have a CDR global score of 0.5, an RBANS score of ≤85 (based upon the Delayed Memory Index score), and an MMSE score between 24 and 30 (inclusive), and they must have had at least 6 years education or work experience. Subjects were to be 50 to 85 years of age at screening. Subjects with medical or neurological conditions other than Alzheimer's disease that may have contributed to the participant's cognitive impairment were excluded. Participants were to be in good health except for Alzheimer's disease.

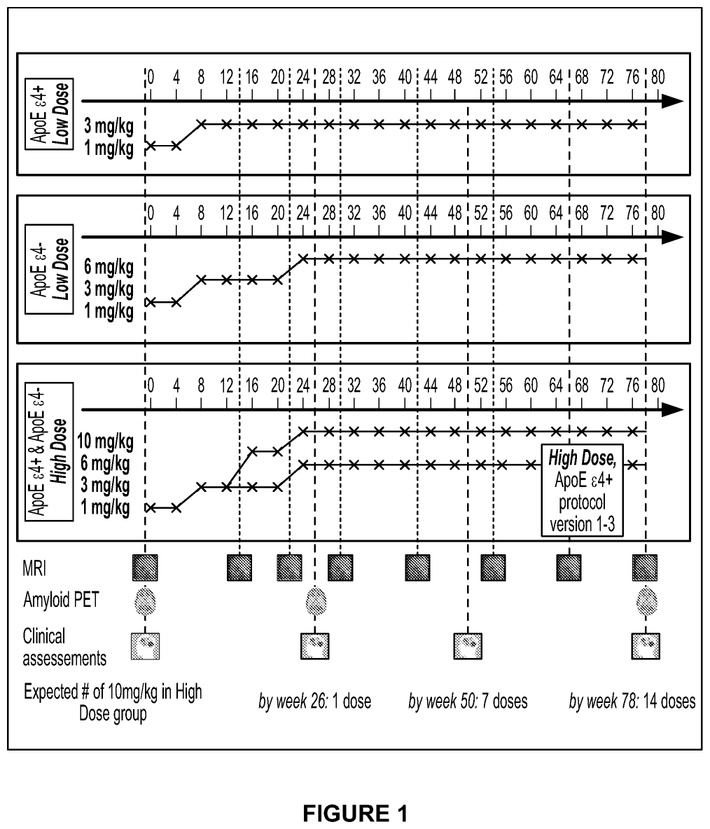

Of note, the Phase 3 protocols also required that subjects undergo ApoE genotyping, given the c4 allele is a major risk factor for Alzheimer's disease. Both ApoE c4 carriers and noncarriers were enrolled in both Phase 3 studies; however, differential dosing based on carrier status was limited to only the aducanumab “low” dose. See, FIG. 1.

### Example 2: Primary Efficacy Endpoint of Study 2

Primary endpoint results for the modified intent to treat (mITT) and Opportunity to Complete (OTC) subjects who had the opportunity to complete the Week 78 visit are summarized in Table 2.
In the high dose group, the advantage of aducanumab over placebo in mean change on the CDR-SB was −0.40 (23% less decline, nominal p=0.0101).
The low dose group also had less decline on the CDR-SB than placebo; however, the differences were smaller than in the high dose group and did not attain statistical significance.

TABLE 2
Change from Baseline in CDR-SB at Week 78: mITT and OTC Populations

| | mITT Population | | | OTC Population | | |
|---|---|---|---|---|---|---|
| | | Diff vs PBO [symbol] | | | Diff vs PBO [symbol] | |
| | | (%) | | | (%) | |
| | | p-value | | | p-value | |
| | PBO decline | Low dose | High dose | PBO decline | Low dose | High d [symbol] e |
| | (N = 548) | (N = 543) | (N = 547) | (N = 313) | (N = 329) | (N = 34) |
| CDR-SB | 1.74 | −0.25 | −0.40 | 1.61 | −0.26 | −0.3 [symbol] |
| | | (−14%) | (−23%) | | (−16%) | (−23% [symbol] |
| | | 0.1171 | 0.0101 | | 0.1342 | 0.030 [symbol] |

[symbol]

difference vs placebo at Week 78. Negative percentage means less progression in the treatment arm.
N: numbers of randomized and dosed subjects that were included in the analysis.
Data source: t-cdr-mmrm-pc-new/Output 6, t-cdr-mmrm-cplt-new/Output 7 [symbol]
indicates data missing or illegible when filed

### Example 3: Secondary Endpoint of Study 2

Secondary efficacy endpoint results for the mITT dataset and the OTC dataset are summarized in Table 3.

For the high dose group, statistically significant differences from placebo (nominal p value <0.05) were observed for all secondary endpoints in both datasets except the MMSE in the mITT dataset. The low dose group did not show statistical significance on any of the 3 secondary endpoints in either the mITT or OTC datasets. However, a small, numeric advantage for low dose over placebo was observed on all endpoints except the MMSE.

TABLE 3
Change from Baseline in MMSE, ADAS-Cog13, and ADCS-ADL-MCI
at Week 78: mITT and OTC Populations

| | mITT Population | | | OTC Population | | |
|---|---|---|---|---|---|---|
| | | Diff vs PBO [symbol] | | | Diff vs PBO [symbol] | |
| | | (%) | | | (%) | |
| | | p-value | | | p-value | |
| | PBO decline | Low dose | High dose | PBO decline | Low dose | High dose |
| | (N = 548) | (N = 543) | (N = 547) | (N = 313) | (N = 329) | (N = 340) |
| MMSE | −3.3 | −0.1 | 0.5 | −3.0 | −0.1 | 0.7 |
| | | (3%) | (−15%) | | (3%) | (−23%) |
| | | 0.6900 | 0.0620 | | 0.8719 | 0.0324 |
| ADAS-Cog13 | 5.171 | 0.747 | −1.395 | 4.835 | −0.484 | −1.219 |
| | | (−14%) | (−27%) | | (−10%) | (−25%) |
| | | 0.1672 | 0.0098 | | 0.4103 | 0.0379 |
| ADCS-ADL- | −4.3 | 0.7 | 1.7 | −4.6 | 0.9 | 2.1 |
| MCI | | (−16%) | (−40%) | | (-20%) | (−46%) |
| | | 0.1556 | 0.0009 | | 0.1171 | 0.0002 |

[symbol]

difference vs placebo at Week 78. Negative percentage means less progression in the treatment arm.
N: numbers of all randomized and dosed subjects that were included in the analysis.
Data source: t-mmse-mmrm-pc-new/Output 12, t-adas-mmrm-pc-new/Output 13, t-adl-mmrm-pc-new/Output 14, 1-mmse-mmrm-cplt-new/Output 15, t-adas-mmrm-cplt-new/Output 16, t-adl-mmrm-cplt-new/Output 17 [symbol]
indicates data missing or illegible when filed

### Example 4: Tertiary Endpoint of Study 2—Brain AR as Measured on PET and Quantified as Standard Uptake Value Ratio (SUVR)

Serial assessment of brain AJ3 plaque levels as measured by Aβ PET and quantified as SUVR was conducted in the subset of subjects participating in the longitudinal Aβ PET sub-study. PET scans in the longitudinal sub-study were performed using the ¹⁸F-florbetapir Aβ PET tracer (except for a small number of subjects in whom another tracer was used). Results for the participants with ¹⁸F-florbetapir PET scans are summarized here.

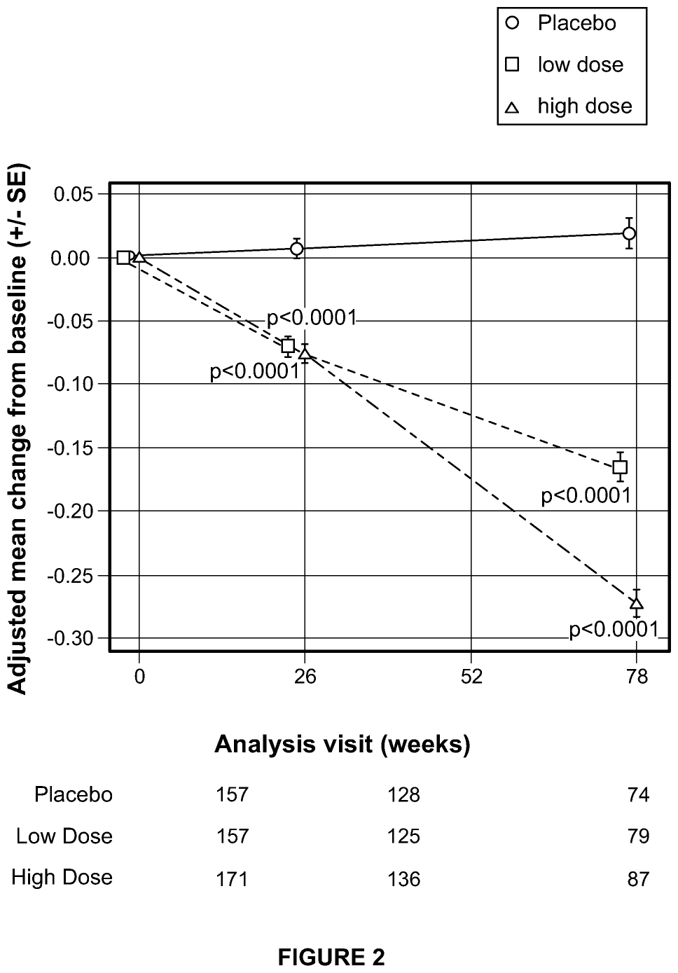

For analyses of aducanumab's effect on brain Af3 plaque levels as measured by PET, a standard uptake value ratio (SUVR; the ratio of radiotracer uptake in regions expected to have Aβ pathology versus a reference region with minimal or no Aβ pathology) was calculated for a composite region of interest comprising the main cortical regions of the brain (parts of the frontal, parietal, lateral temporal, sensorimotor, anterior, and posterior cingulate) with whole cerebellum serving as the reference region [Ostrowitzki et al., Alzheimers Res. Ther.8; 9(1):95 (2017); Chiao et al., J Nucl Med. 60(1):100-106 (2019); Sevigny, Nature 537(7618):50-6 (2016)]. This SUVR on the composite of regions was used as the primary endpoint for Aβ PET analysis. A negative change from baseline in the composite SUVR indicates a reduction in Aβ plaque level and a negative treatment difference (aducanumab minus placebo) favors aducanumab. FIG. 2, depicts the time- and dose-dependent reductions in brain Aβ levels. At Week 26, coincident with the end of the titration phase, the adjusted mean change from baseline in Aβ PET composite SUVR was −0.070 and −0.076 in the low and high dose groups, respectively, compared with 0.007 in the placebo group. Due to the similarity of dosing during the titration phase, separation between the low and the high dose groups was not anticipated. At Week 78, the adjusted mean change from baseline in βB PET composite SUVR was −0.165 and −0.272 in the low and high dose groups, respectively, compared with 0.019 in the placebo group.

### Example 5: Primary Efficacy Endpoint of Study 1

Results of the mITT and OTC analyses of the primary endpoint show that aducanumab high dose did not reduce decline compared with placebo (Table 4). The low dose group did not show nominal statistical significance on the primary endpoint. However, a small, numeric advantage for the low dose over placebo was observed. This difference was similar in magnitude to the difference between low dose and placebo in Example 1.

TABLE 4
Change from Baseline in CDR-SB at Week 78: mITT and OTC Populations, April Dataset

| | mITT Population | | | OTC Population | | |
|---|---|---|---|---|---|---|
| | | Diff vs PBO [symbol] | | | Diff vs PBO [symbol] | |
| | | (%) | | | (%) | |
| | | p-value | | | p-value | |
| | PBO decline | Low dose | High dose | PBO decline | Low dose | High dose |
| | (N = 545) | (N = 547) | (N = 555) | (N = 369) | (N = 370) | (N = 345) |
| CDR-SB | 1.55 | −0.18 | 0.03 | 1.45 | −0.11 | 0.08 |
| | | (−12%) | (2%) | | (−8%) | (6%) |
| | | 0.2362 | 0.8252 | | 0.4887 | 0.6266 |

[symbol]

difference vs placebo at Week 78. Negative percentage means less progression in the treatment arm.
N: numbers of all randomized and dosed subjects that were included in the analysis.
Data source: t-cdr-mmrm-pc-new/Output 6, t-cdr-mmrm-cplt-new/Output 7 [symbol]
indicates data missing or illegible when filed

### Example 6: Secondary Efficacy Endpoint of Study 1

Results of the mITT and OTC analyses of the secondary endpoints show no statistically significant differences in decline on MMSE, ADAS-Cog13, or ADCS-ADL-MCI compared with placebo (Table 5). However, small numeric advantages for the low dose over placebo was observed on these endpoints. For ADAS-Cog13, or ADCS-ADL-MCI, results for the high dose group were similar to the low dose group.

TABLE 5
Change from Baseline in MMSE, ADAS-Cog13, and ADCS-ADL-MCI
at Week 78: Study 301, mITT and OTC Populations, April Dataset

| | mITT Population | | | OTC Population | | |
|---|---|---|---|---|---|---|
| | | Diff vs PBO [symbol] | | | Diff vs PBO [symbol] | |
| | | (%) | | | (%) | |
| | | p-value | | | p-value | |
| | PBO decline | Low dose | High dose | PBO decline | Low dose | High dose |
| | (N = 545) | (N = 547) | (N = 555) | (N = 369) | (N = 370) | (N = 345) |
| MMSE | −3.5 | 0.2 | −0.1 | −3.2 | 0.1 | −0.4 |
| | | (−6%) | (3%) | | (−3%) | (13%) |
| | | 0.4875 | 0.7961 | | 0.7406 | 0.2367 |
| ADAS-Cog13 | 5.171 | −0.590 | −0.605 | 4.813 | −0.035 | −0.091 |
| | | (−11%) | (−12%) | | (−1%) | (−2%) |
| | | 0.2475 | 0.2446 | | 0.9495 | 0.8735 |
| ADCS-ADL- | −3.8 | 0.7 | 0.7 | −3.3 | 0.4 | 0.4 |
| MCI | | (−18%) | (−18%) | | (−12%) | (−12%) |
| | | 0.1345 | 0.1520 | | 0.4343 | 0.4051 |

[symbol]

difference vs placebo at Week 78. Negative percentage means less progression in the treatment arm.
N: numbers of all randomized and dosed subjects that were included in the analysis.
Data source: t-mmse-mmrm-pc-new/Output 12, t-adas-mmrm-pc-new/Output 13, t-adl-mmrm-pc-new/Output 14, 1-mmse-mmrm-cplt-new/Output 15, t-adas-mmrm-cplt-new/Output 16, t-adl-mmrm-cplt-new/Output 17 [symbol]
indicates data missing or illegible when filed

### Example 7: Tertiary Endpoint of Study 1—Brain Aβ as Measured on PET and Quantified as Standard Uptake Value Ratio (SUVR)

As in the study of Example 3, a longitudinal Aβ PET sub-study was conducted using the ¹⁸F-florbetapir Aβ PET tracer.

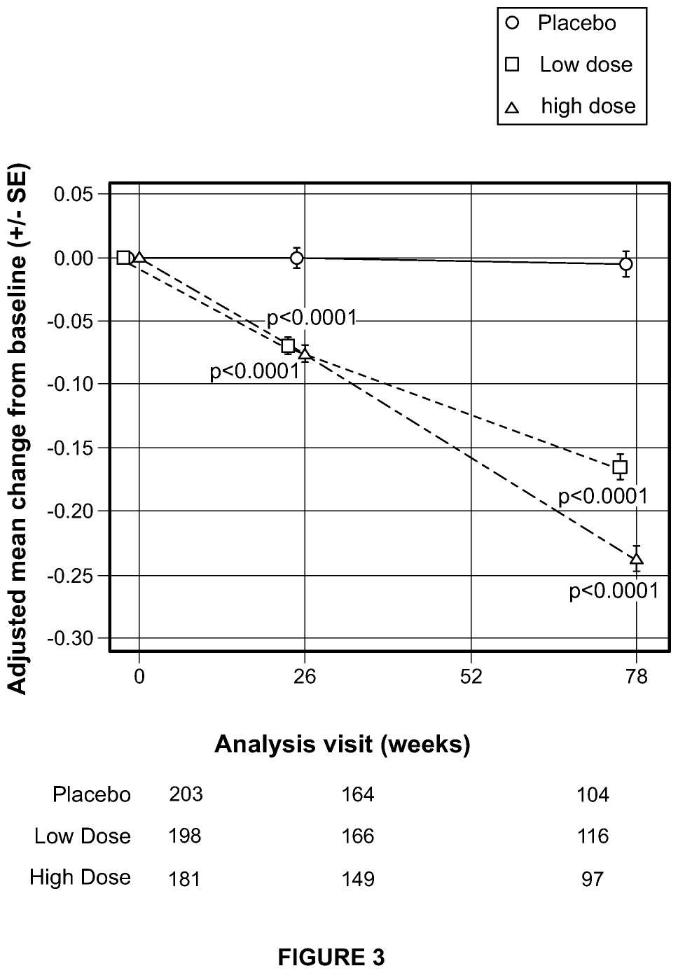

As can be seen in FIG. 3, aducanumab resulted in time- and dose-dependent reductions in brain Aβ levels. At Week 26, coincident with the end of the titration phase, the adjusted mean change from baseline in Aβ PET composite SUVR was −0.066 in both the aducanumab low and high dose groups, compared with −0.002 in the placebo group. Due to the similarity of dosing during the titration phase, separation between the low and the high dose groups was not anticipated. At Week 78, the adjusted mean change from baseline in Aβ PET composite SUVR was −0.168 and −0.238 in the aducanumab low and high dose groups, respectively, compared with −0.005 in the placebo group.

### Example 8: CSF Levels of p-Tau in Studies 1 and 2

Cerebrospinal fluid (CSF) was collected at baseline and at Week 78 in a subset of participants at select sites. CSF levels of p-Tau_{181P}, and total tau were measured using the Lumipulse® G immunoassays (Fujirebio, Malvern, Pa., USA).

CSF levels of p-Tau are correlated with neocortical neurofibrillary tangles [Buerger, Brain, 129(Pt 11):3035-41 (2006)] as well as Tau PET imaging [Gordon, Brain 139(Pt8):2249-60 (2016)]. Elevated CSF levels of p-Tau have been reported to be specific to Alzheimer's disease [Olsson, Lancer Neurol., 15(7):673-684 (2016)]. Analysis of publicly available Alzheimer's Disease Neuroimaging Initiative (ADNI) data indicate an expected annual increase in CSF p-Tau of approximately 2%.

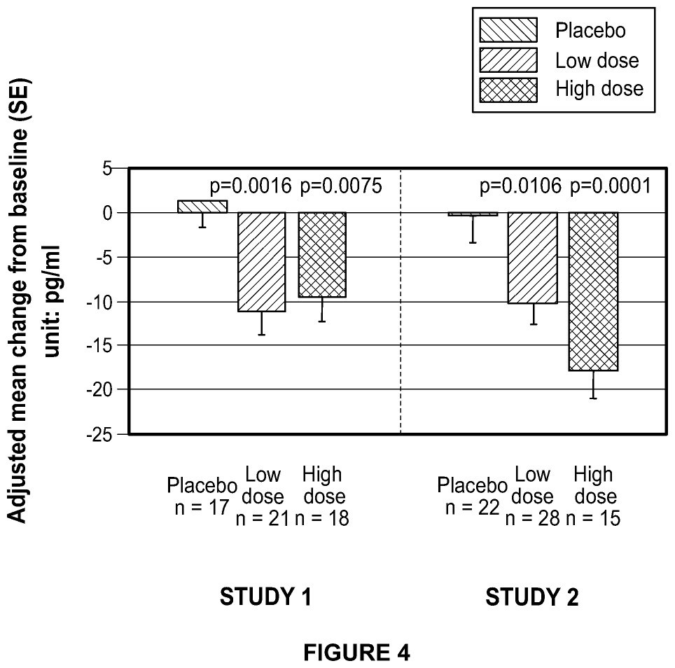

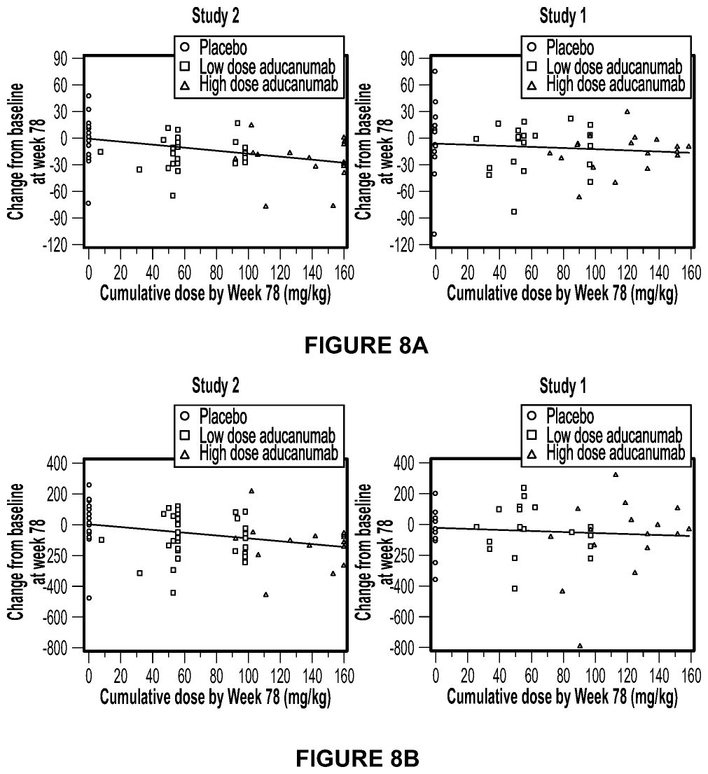

Consistent with the effect of aducanumab on Tau PET, a statistically significant reduction in CSF p-Tau levels were observed in Studiesl and 2, with a dose proportional response in Study 2 (see, FIGS. 4 and 8A).

### Example 9: t-Tau Levels in Studies 1 and 2

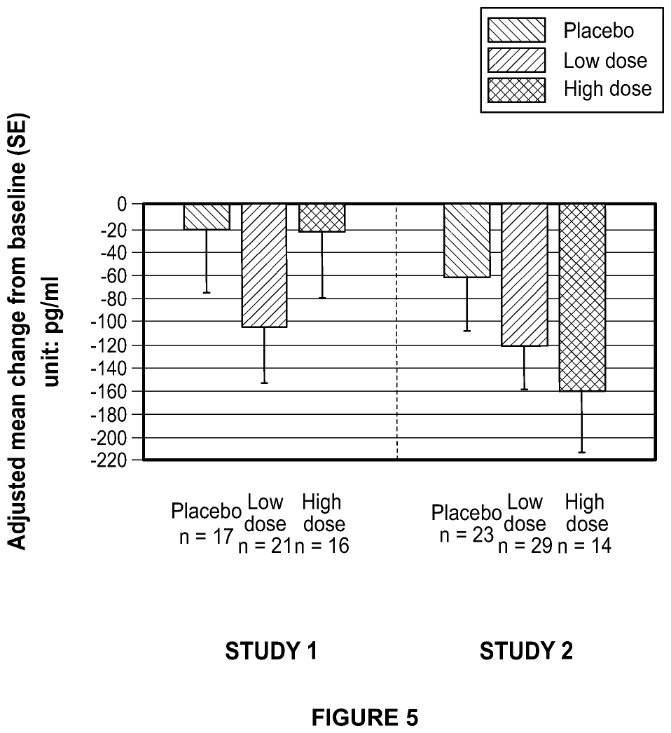

In contrast to p-Tau, elevated CSF t-Tau levels have been reported in multiple neurodegenerative diseases as well as in traumatic brain injury and stroke [Jack et al., Alzheimers Dement, 14(4):535-562 (2018)] and is considered to reflect non-specific neuronal and axonal degeneration in the brain [Blennow et al., Nat. Rev. Neurol., 6(3):131-44 (2010)]. As seen in FIGS. 5 and 8B, aducanumab produced a numeric reduction in CSF t-Tau levels in Study 1 and Study 2, with a dose proportional response in Study 2.

### Example 10: Mean Aβ PET Composite SUVR Time Profiles for Patients with >10 Doses of 10/mg/kg at Steady State

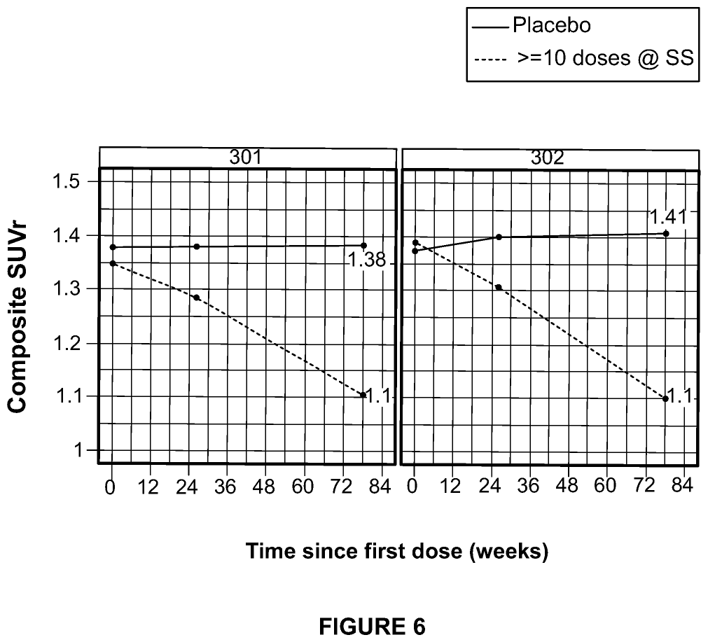

FIG. 6 presents the mean brain Aβ PET composite SUVR-time profiles of those subjects from Studies 1 and 2 who belong to the to the groups with >10 state dosing intervals at 10 mg/kg with the respective placebo group Aβ PET composite SUVR profiles. The mean Aβ PET composite SUVR profiles of the two groups of interest from Studies 1 and 2 have very similar shape and the mean values at Week 78 are identical.

### Example 11: Mean CDR-SB Time Profiles of Those Individuals from Studies 1 and 2

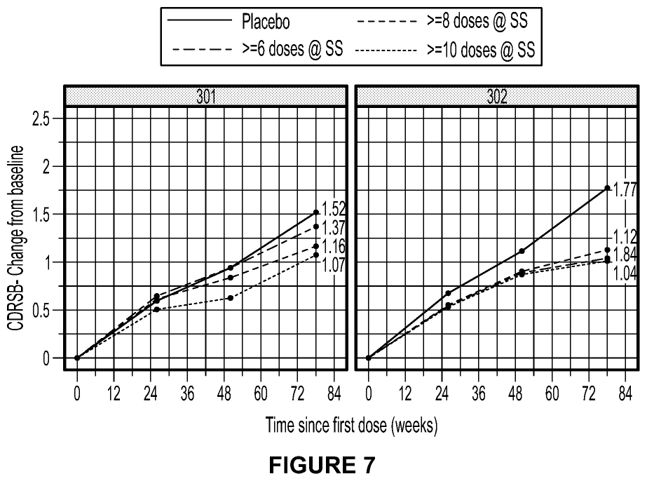

FIG. 7 presents the mean CDR-SB time profiles of patients in Studies 1 and 2 with ≥6 steady-state dosing intervals at 10 mg/kg (sample size Study 1: n=241 and Study 2:n=257), ≥8 steady state dosing intervals at 10 mg/kg (sample size Study 1: n=186 and Study 2:n=194), and ≥10 steady-state dosing intervals at 10 mg/kg (sample size Study 1: n=116 and Study 2:n=147). The respective placebo group CDR-SB profiles are also shown.

In Study 2, the various dosing groups had similar CDR-SB response. In Study 1, differences from placebo increased as the number of uninterrupted 10 mg/kg doses increased. This result implies that much of the difference between studies may be due to the subjects who had suboptimal dosing of <10 uninterrupted doses of 10 mg/kg.

### Example 12: Tau PET Study

Tau PET imaging using ¹⁸F-MK-6240 ligand was performed in a subset of participants at select sites at screening and at Week 78. Composite Standardized Uptake Value Ratios (SUVRs) for medial temporal, temporal, and frontal regions were calculated using cerebellar cortex as the reference region. Due to the small sample size, all the analyses were conducted using pooled data from both studies.

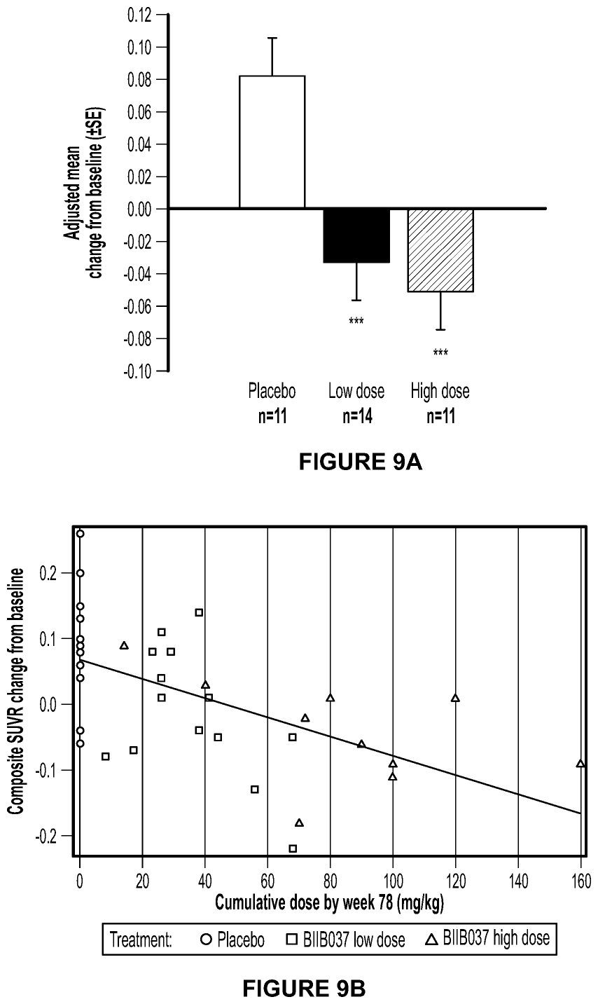

The tau PET study pooled data from both studies (n=37) and analyzed tau deposition using ¹⁸F-MK-6240 as the tau ligand. Participants treated with aducanumab showed a statistically significant and dose-dependent reduction in tau SUVR levels in the medial temporal composite brain region versus placebo (P=0.0012 for low-dose and P=0.0005 for high-dose) (FIG. 9A). Tau PET medial temporal composite SUVR change from baseline was correlated with cumulative dose by Week 78 (FIG. 9B). ¹⁸F-MK-6240 has been shown as a suitable PET tau tracer for in vivo imaging as well as characterization and quantification of neurofibrillary tau tangles and aggregates in Alzheimer's disease; see, e.g., Pascoal et al. Alzheimer's Research & Therapy (2018) 10:74 and Betthauser et al., J. Nucl. Res. Med. 60 (2019), 93-99. Thus, together with findings in the PRIME study illustrated in Example 14 below. the results of the tau PET study clearly demonstrate that aducanumab, in particular at high dose and in dose regimes disclosed herein is capable of reducing the number of tau tangles in the brain of Alzheimer's disease patients.

In summary, PET and CSF Biomarker studies showed that aducanumab reduced Aβ , as well as tau pathology and neurodegeneration in participants with early Alzheimer's disease. This was the first demonstration in a Phase 3 trial that modification of underlying disease pathology was associated with statistically significant slowing of clinical decline. Therefore, the results from Study 1 and Study 2 provide important validation that clearance of Aβ can lead to clinical benefit.

### Example 13: Safety

The incidence of adverse events occurring during the placebo-controlled study period was comparable between placebo and aducanumab groups across both studies. In Study 2, 93.1% of participants in high-dose, 89.5% in low-dose, and 87.4% in the placebo group had an adverse event; in Study 1, 90.1% of participants in high-dose, 90.0% of participants in low-dose, and 86.5% in the placebo group had an adverse event. Except for amyloid-related imaging abnormalities (ARIA), the incidence and nature of adverse events were consistent with the diagnosis of Alzheimer's disease and the expected comorbidities for the age of the study population (median years of age: 70.7 in Study 2 and 70.1 in Study 1). There were 16 fatal events in the placebo-controlled period between the two studies, 11 in aducanumab and 5 in placebo-treated participants. The reported causes of death were consistent with Alzheimer's disease or underlying comorbidities such as cardiovascular disease.

As shown in Table 6, the most common adverse events with an incidence above 10% in any treatment group were ARIA-Edema (ARIA-E), brain microhemorrhages (termed ARIA-H microhemorrhage in the studies), headache, nasopharyngitis, fall, localized superficial siderosis (termed ARIA-H superficial siderosis in the studies), and dizziness. ARIA-E was the most common adverse event in aducanumab-treated participants. An increased incidence of brain microhemorrhages and localized superficial siderosis was observed in aducanumab-treated participants compared with placebo. In these participants, microhemorrhages and localized superficial siderosis were frequently concurrent with ARIA-E. In the absence of ARIA-E, the incidence of brain microhemorrhages and localized superficial siderosis was comparable between the aducanumab and placebo groups.

TABLE 6
Summary of Advserse Events

| | Study 2 [symbol] | | | | | | Study 1 [symbol] | | | | | |
|---|---|---|---|---|---|---|---|---|---|---|---|---|
| Event, n (%) | Placebo | | Low dose | | High dose | | Placebo | | Low dose | | High [symbol] | |
| Safety MRI population | n = 544 | | n = 537 | | n = 541 | | n = 532 | | n = 545 | | n = 5 [symbol] | |
| ARIA-Edema | 13 | (2.4) | 140 | (26.1) | 188 | (34.8) | 16 | (3.0) | 141 | (25.9) | 199 | ( [symbol] 9) |
| Brain microhemorrhage | 37 | (6.8) | 87 | (16.2) | 108 | (20.0) | 34 | (6.4) | 89 | (16.3) | 104 | ( [symbol] 8) |
| Brain microhemorrhage in | 35 | (6.6) | 30 | (7.6) | 32 | (9.1) | 32 | (6.2) | 24 | (5.9) | 21 | ( [symbol] ) |
| participants without ARIA-E | | | | | | | | | | | | |
| Localized superficial siderosis | 14 | (2.6) | 52 | (9.7) | 73 | (13.5) | 10 | (1.9) | 51 | (9.4) | 89 | (11) |
| Localized superficial siderosis | 9 | (1.7) | 9 | (2.3) | 7 | ( 2.0) | 6 | (1.2) | 7 | (1.7) | 5 | (1) |
| in participants without ARIA E | | | | | | | | | | | | |
| Safety population | n = 547 | | n = 544 | | n = 547 | | n = 540 | | n = 549 | | n = 5 [symbol] | |
| Headache | 84 | (15.4) | 110 | (20.2) | 107 | (19.6) | 81 | (15.0) | 99 | (18.0) | 115 | ( [symbol] 6) |
| Nasopharyngitis | 91 | (16.6) | 71 | (13.1) | 89 | (16.3) | 64 | (11.9) | 65 | (11.8) | 68 | (12) |
| Fall | 71 | (13.0) | 68 | (12.5) | 76 | (13.9) | 57 | (10.6) | 80 | (14.6) | 86 | (14) |
| Dizziness | 44 | (8.0) | 42 | (77) | 55 | (10.1) | 54 | (10.0) | 49 | (8.9) | 54 | ( [symbol] ) |

[symbol]

indicates data missing or illegible when filed

The majority of participants with ARIA did not exhibit symptoms during ARIA (Study 2: 80.3% in high-dose, 78.7% in low-dose, 96.4% in placebo; Study 1: 71.2% in high-dose, 83.6% in low-dose, 94.5% in placebo). Symptoms reported in the setting of ARIA were transient and included headache, confusion, and dizziness. The majority of ARIA-E episodes resolved within 12 weeks and the majority of participants with ARIA either remained on treatment without interruption or resumed treatment after a temporary suspension. Specifically, a total of 65 participants in Study 2 (7.0% in high-dose, 4.8% in low-dose, 0.2% in placebo) and 73 participants in Study 1 (7.2% in high-dose, 5.1% in low-dose, 0.9% in placebo) permanently discontinued study treatment due to ARIA.

Permanent treatment discontinuation in participants with ARIA may be required if the subject develops any of the following:
ARIA-E accompanied by serious clinical symptoms except for “other medically important event”*
Symptomatic ARIA-H (microhemorrhages) with serious clinical symptoms except for “other medically important event”*
Symptomatic ARIA-H (superficial siderosis) with or serious clinical symptoms except for “other medically important event”*
ARIA-H with ≥10 microhemorrhages and/or ≥2 focal areas of superficial siderosis.
Any new incident macrohemorrhage (defined as >1 cm in diameter on T2* sequence).
The subject becomes pregnant. Study treatment must be discontinued immediately and pregnancy must be reported
The subject withdraws consent to continue study treatment.
The subject experiences a medical emergency that necessitates permanent discontinuation of study treatment or unblinding of the subject's treatment assignment.
The subject experiences an AE that does not resolve or requires continued treatment that meets exclusionary criteria.
The subject experiences a severe infusion reaction.
At the discretion of the Investigator for medical reasons.
At the discretion of the Investigator or Sponsor for noncompliance.

A subject who discontinues treatment remains in the study, and attends a FU Visit 18 weeks after the final dose, and immediately continues protocol-required tests and assessments at a subset of the clinic visits until the end of the study per the schedule of events or until the subject withdraws consent.
*=“Other medically important event” includes those that are life-threatening (in the opinion of the Investigator), require inpatient hospitalization or prolongation of existing hospitalization, and/or result in persistent or significant disability/incapacity or a congenital anomaly/birth defect.

In sum, the most common adverse events were ARIA-E and headache. The majority of ARIA episodes were transient and asymptomatic.

### Example 14: Alzheimer Disease Neuropathology in a Patient Treated with Aducanumab

### Case Presentation

The patient was an 84-year-old woman who was diagnosed with mild probable AD in 2013. Her APOE genotype was E3/E3. Her past medical history was pertinent for coronary artery disease (history of coronary artery stent), hypertension, hyperlipidemia and depression.

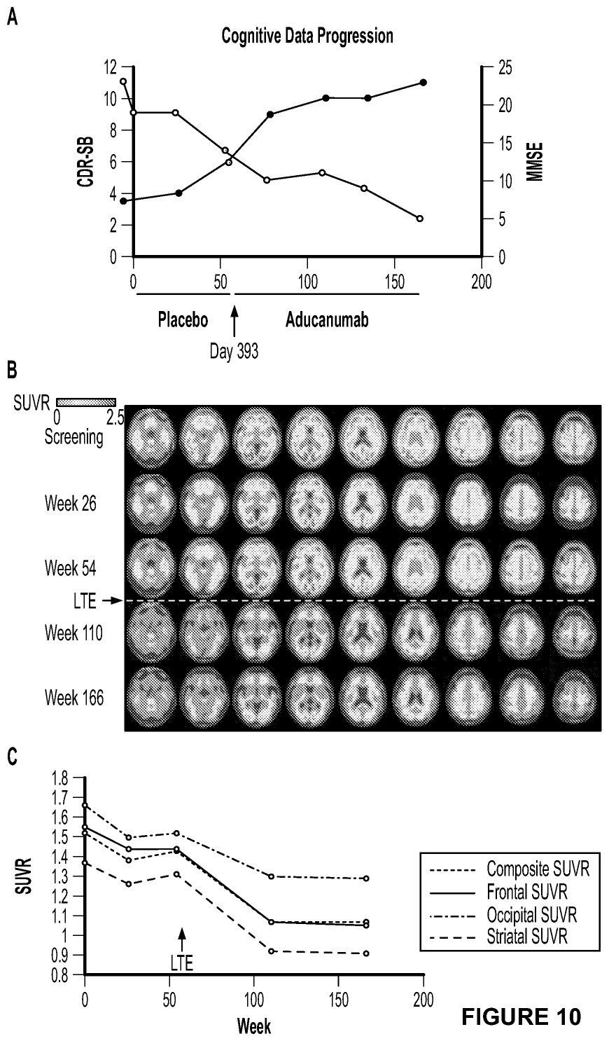

Her medications included Exelon patch and Namenda. Screening cognitive data for the Trial demonstrated a CDR-SB score of 3.5 and MMSE of 23, corresponding to mild cognitive impairment (FIG. 10A). An amyloid-PET (florbetapir) scan demonstrated All plaque throughout the cerebral cortex and striatum (FIGS. 10B-C). She was randomized to the placebo arm of the Phase lb PRIME Trial during which she received 14 IV infusions of placebo from 5-1-2013 to 4-30-2014. The patient demonstrated rapid progression of her cognitive impairment, with 54-week scores of 6 on the CDR-SB and 15 on the MMSE corresponding to mild-to-moderate dementia (FIG. 10A).

The patient then enrolled in the LongTerm Extension (LTE), during which she received 2 monthly doses of aducanumab 3 mg/kg IV followed by 30 monthly 6 mg/kg IV doses between 6- 2-2014 to 11-10-2016. During this time, all surveillance MRIs were negative for amyloid-related imaging abnormalities (ARIA). Amyloid PET scan studies at 110 and 166 weeks, 56 and 112 weeks following the start of aducanumab dosing, respectively, demonstrated robust standardized uptake value ratio (SUVR) reductions in the frontal, temporal and parietal cortices and striatum (FIGS. 10B-C). SUVR signal declined in the occipital cortex during aducanumab dosing, but remained elevated compared to the other regions. Despite robust Aβ plaque reduction, the patient continued to decline cognitively with moderate to severe dementia (CDR-SB of 11; MMSE of 5/30). She entered skilled nursing care in November 2016 at which time she discontinued aducanumab. The patient died 4 months later in March, 2017. A brain donation autopsy was performed at Yale University following a 17-hour post-mortem interval.

### Neuropathologic Examination

Gross brain examination revealed a brain weight of 1,000 grams. The leptomeninges showed no hemorrhage or hemosiderin. There was symmetrical cortical atrophy involving the frontal, temporal and parietal lobes. Coronal sections demonstrated hippocampal atrophy. The substantia nigra was normally pigmented. There were no recent and remote infarcts or parenchymal hemorrhages. According to NIA/AA consensus guidelines, tissue sections examined at Yale confirmed the presence of Alzheimer disease neuropathologic changes: Aβ plaques were observed in neocortex and hippocampus (Thal Phase 2), NFTs were observed in sections of association neocortex (Braak stage V) and sparse neocortical NPs were observed on modified Bielschowsky stained sections (CERAD score 1). The composite NIA/AA ABC score was A1, B3 C1, consistent with “Low AD Neurpathologic Changes”. There was no significant glial tauopathy, no ballooned neurons or other neuropathologic signs of non-Alzheimer tauopathy. The neuropathologic examination was also negative for Lewy bodies and TDP-43 proteinopathy, hippocampal sclerosis (LATE) and microinfarcts.

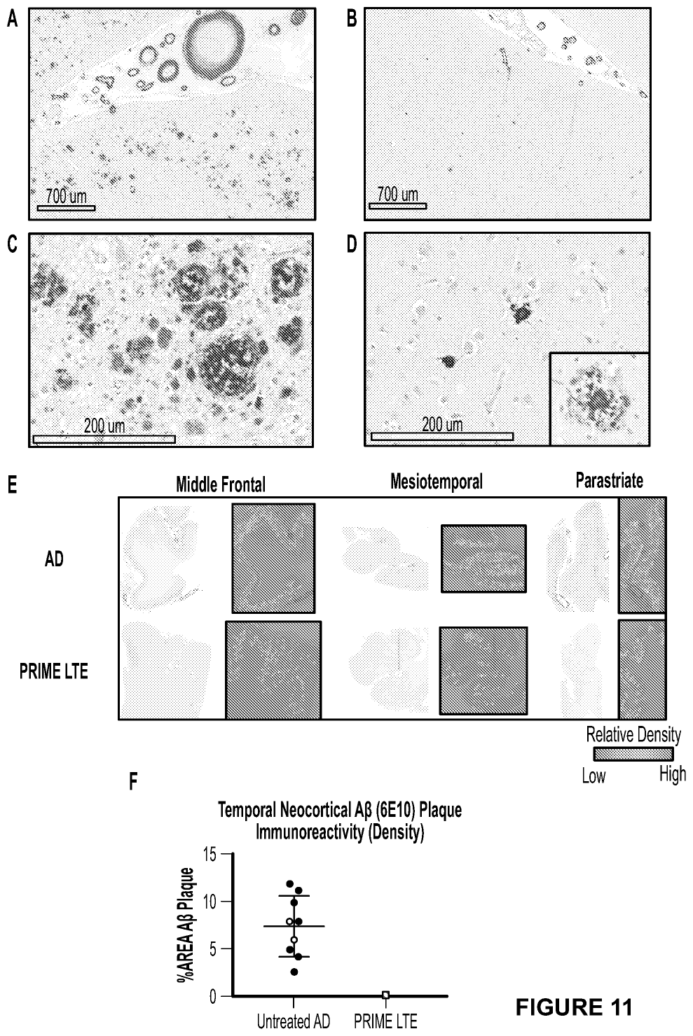

Aβ immunohistochemical stains (antibody 6E10) revealed frequent Aβ plaques in sections from untreated HIGH AD cases (FIG. 11A, 11C). In contrast, cortical sections from the LTE Patient (FIG. 11B, 11D) showed sparse Aβ plaques. The residual plaques in sections from the LTE Patient were comprised predominantly of dense-cores lacking peripheral halos of non-compact Aβ (FIG. 11D). Where peripheral halos of non-compact Aβ persisted, particularly in sections of the parastriate cortex, they demonstrated a moth-eaten appearance with conspicuous reactive microglia (FIG. 11D, inset). Cortical Aβ plaque whole slide image (WSI) heatmaps generated in Visiopharm demonstrated clearance of Aβ plaques throughout sections of frontal, temporal and occipital cortex in the LTE Patient compared to high plaque densities in sections from an untreated HIGH AD case (FIG. 11E). The highest levels of residual Aβ plaque immunoreactivity were present in the parastriate cortex of the occipital lobe (FIG. 11E, right panels), in agreement with the Amyloid-PET SUVR data. Sections of the basal ganglia and midbrain were devoid of Aβ plaque. Temporal neocortical Aβ plaque densities were compared between the LTE patient and a cohort of HIGH AD patients who did not receive aducanumab (FIG. 11F), revealing that Aβ plaques were markedly lower in the LTE Patient. Taken together, these ex vivo neuropathologic findings confirm the robust Aβ plaque clearance in the LTE Patient demonstrated with florbetapir-PET.

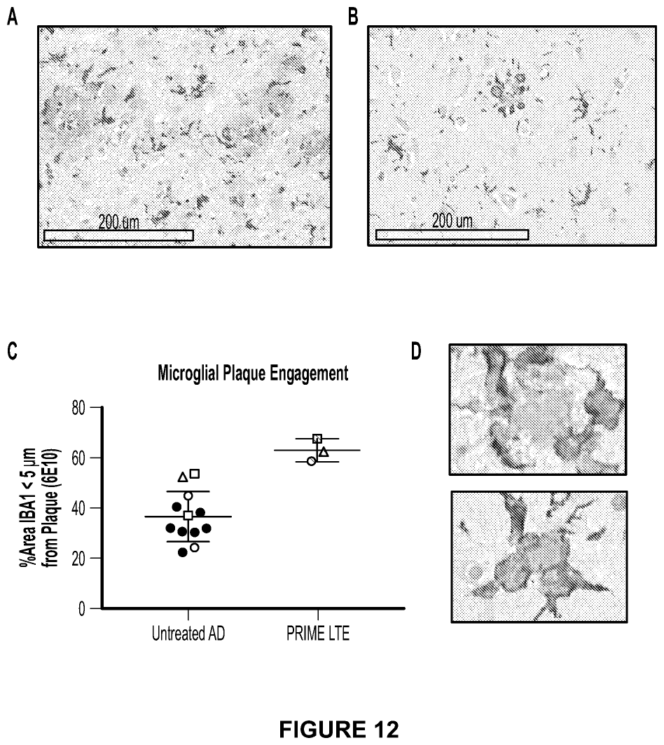

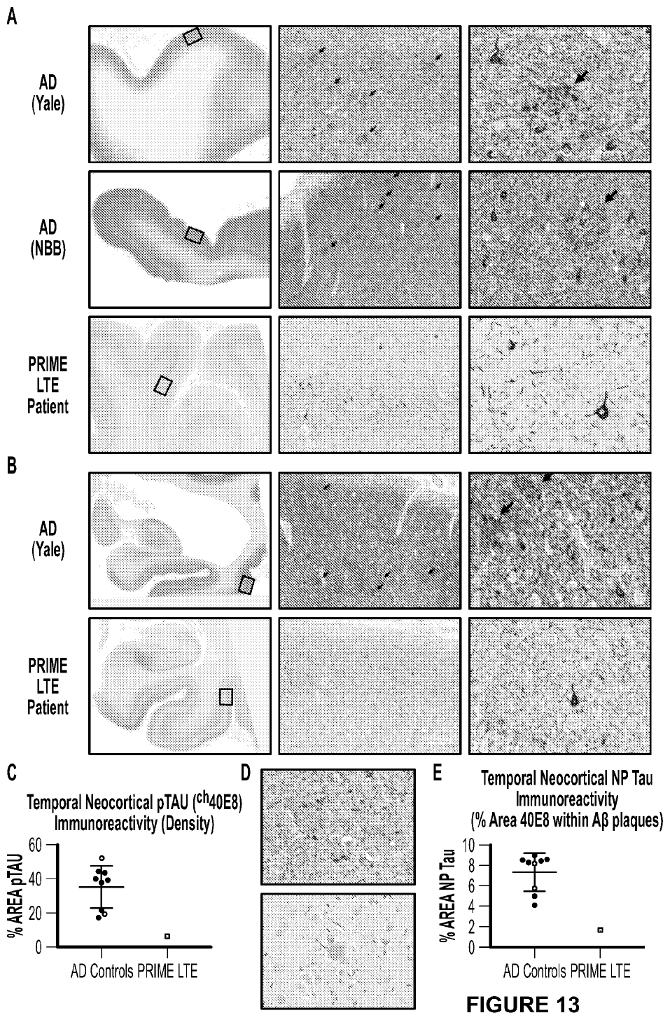

A dual 6E10/1BA1 immunohistochemistry assay was employed to examine microglial reactivity to residual Aβ plaques. Compared to sections from an untreated HIGH AD case (FIG. 12A), residual and moth-eaten plaques in the LTE Patient appeared to demonstrate greater association of microglia with highly reactive amoeboid morphology (FIG. 12B). A WSI analysis algorithm that was designed to segment microglial IBA1 immunoreactivity within 5-μm radii from Aβ plaque edges demonstrated higher microglial plaque engagement in the LTE Patient compared to the cohort of untreated HIGH AD patients (FIG. 12C). High-power views of Aβ plaques in the LTE Patient demonstrated Aβ plaque surrounded by microglial processes and Aβ within the plasma membrane borders of amoeboid microglia (FIG. 12D). Phosphorylated TAU^{Ser202,Thr205} (pTau, 40E8) immunohistochemical stains performed on [symbol] association neocortex, indicative of Braak stage V/VI (NIA/AA stage B3) neurofibrillary degeneration. However, compared to sections from untreated HIGH AD cases, sections of frontal and occipitotemporal neocortex from the LTE Patient were remarkable for lower densities of pTau immunoreactivity (FIG. 13A, FIG. 13B). A whole-slide image analysis algorithm designed to segment and quantify somatic and neuropil thread pTau immunoreactivity demonstrated lower neocortical pTau density in the LTE patient compared to a range of untreated HIGH AD cases (FIG. 13C). A dual Aβ, pTau (40E8) immunohistochemistry assay used to segment neuritic plaque pTau (NP Tau), pTau immunoreactive dystrophic neurites around Aβ plaques that propagate proteopathic Tau seeds in AD, demonstrated abundant NP Tau in sections from untreated HIGH AD patients (FIG. 13D, top panel) but residual plaques without NP Tau in the LTE patient (FIG. 13D, lower panel). The mean density of NP Tau in the LTE patient was lower than the range of NP Tau densities in control HIGH AD specimens (FIG. 13E).

In summary, these are the first neuropathologic data from a patient with AD who was treated with aducanumab. The neuropathologic findings corroborate florbetapir-PET data demonstrating the removal of Aβ plaques, demonstrate microglial engagement and phagocytosis of Aβ plaques and provide evidence of pTau neuropathology reduction consistent with Tau-PET and CSF pTau biomarker assay data in Study 1 and Study 2.

### Example 15: Final Analyses for the Placebo-Controlled Period for Study 1 and Study 2

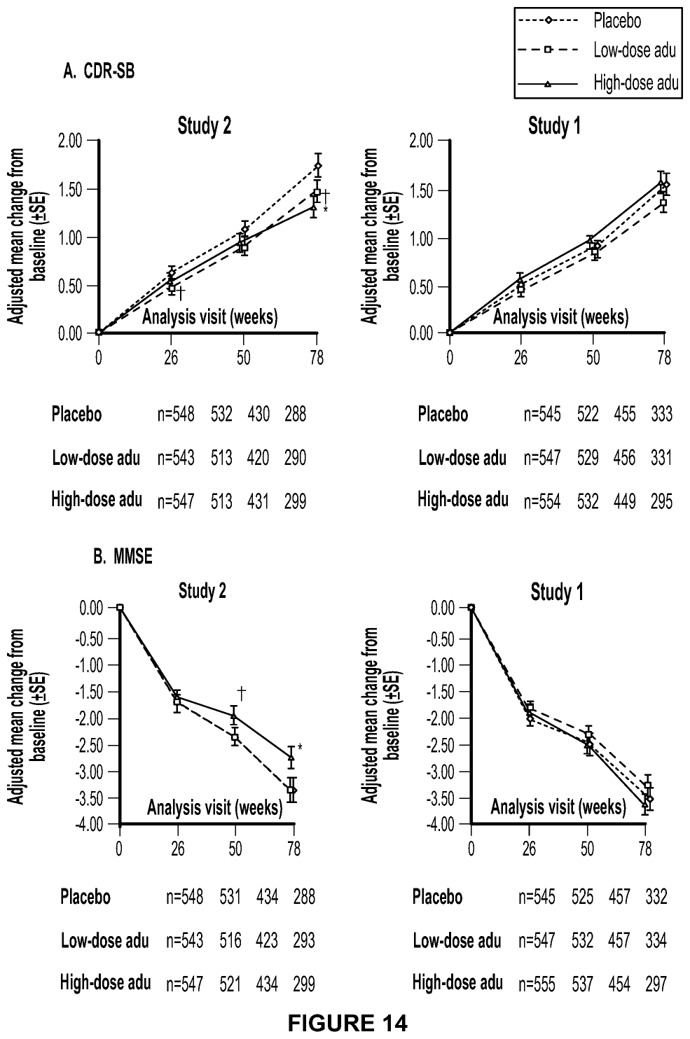

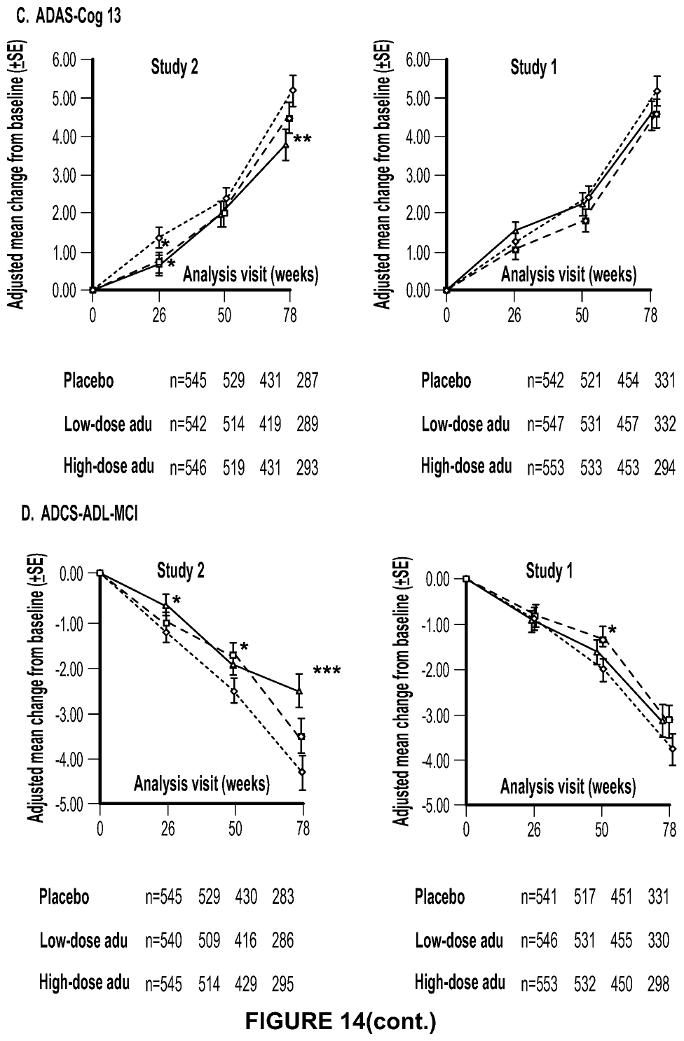

The primary endpoint was met in Study 2. High-dose aducanumab versus placebo demonstrated a mean difference in change from baseline in CDR-SB score at Week 78 was −0.39 (95% confidence interval, −0.69 to −0.09; P=0.012), a decrease of 22% (Table 7, FIG. 14A-D). High-dose aducanumab also showed (P<0.05) slower rates of decline versus placebo in MMSE (−18%), ADAS-Cog13 (−27%), and ADCS-ADL-MCI (−40%) scores. The low-dose aducanumab arm yielded no statistically significant differences versus placebo.

The primary endpoint was not met in Study 1. With high-dose aducanumab versus placebo, the mean difference in change from baseline in CDR-SB score at Week 78 was 0.03 (95% confidence interval, −0.26 to 0.33; P=0.833), an increase of 2% (Table 7, FIG. 14A-D). Changes versus placebo in MMSE (3%), ADAS-Cog (−11%), and ADCS-ADL-MCI (−18%) scores were not statistically significant. Results for the low-dose arm were consistent with those from Study 2.

TABLE 7
Primary and secondary endpoints at Week 78

| | Study 2 | | | | | Study 1 | | | | |
|---|---|---|---|---|---|---|---|---|---|---|
| | Placebo | Difference vs. placebo (%)* | | | | Placebo | Difference vs. placebo (%)* | | | |
| | decline ± | 95% CI; P-value | | | | decline ± | 95% CI; P-value^{†} | | | |
| | SE | Low dose | | High dose | | SE | Low dose | | High dose | |
| Endpoint | n = 548 | n = 543 | | n = 547 | | n = 545 | n = 547 | | n = 555 | |
| CDR-SB^{†} | 1.74 ± 0.11 | −0.26 | (−15%) | −0.39 | (−22%) | 1.56 ± 0.11 | −0.18 | (−12%) | 0.03 | (2%) |

| | | | |
|---|---|---|---|
| −0.57, 0.04; 0.090 | −0.69, −0.09; 0.012 | −0.47, 0.11; 0.225 | −0.26, 0.33; 0.833 |

| MMSE^{‡} | −3.3 ± 0.2 | −0.1 | (3%) | 0.6 | (−18%) | −3.5 ± 0.2 | 0.2 | (−6%) | −0.1 | (3%) |
|---|---|---|---|---|---|---|---|---|---|---|
| | | −0.7, 0.5; 0.758 | | 0.0, 1.1; 0.049 | | | −0.3, 0.7; 0.479 | | −0.6, 0.5; 0.811 | |
| ADAS-Cog 13^{§} | 5.16 ± 0.40 | −0.70 | (−14%) | −1.40 | (−27%) | 5.14 ± 0.38 | −0.58 | (−11%) | −0.59 | (−11%) |

| | | | |
|---|---|---|---|
| −1.76, 0.36; 0.196 | −2.46, −0.34; 0.010 | −1.58, 0.42; 0.254 | −1.61, 0.43; 0.258 |

| | | | | | | | | | | |
|---|---|---|---|---|---|---|---|---|---|---|
| ADCS-ADL-MCI^{¶} | −4.3 ± 0.4 | 0.7 | (−16%) | 1.7 | (−40%) | −3.8 ± 0.3 | 0.7 | (−18%) | 0.7 | (−18%) |

| | | | |
|---|---|---|---|
| −0.3, 1.7; 0.151 | 0.7, 2.7; <0.001 | −0.2, 1.6; 0.123 | −0.2, 1.6; 0.151 |

The analyses were performed in the intention-to-treat population. A mixed model for repeated measures was used to assess CDR-SB, MMSE, ADAS-Cog13, and ADCS-ADL-MCI scores, with fixed effects of treatment, categorical visit, treatment-by-visit interaction, baseline score, baseline score-by-visit interaction, baseline MMSE score (same as baseline score in the MMSE model), Alzheimer’s disease symptomatic medication use at baseline, region, and ApoE ε4 status (carrier and noncarrier).
*Difference versus placebo at Week 78. Negative percentage means less progression in the treated arm.
^{†}CDR-SB scores range from 0 to 18, with higher scores indicating greater impairment.
^{‡}MMSE scores range from 0 to 30, with lower scores indicating greater impairment.
^{§}ADAS-Cog13 scores range from 0 to 85, with higher scores indicating greater impairment.
^{¶}ADCS-ADL-MCI scores range from 0 to 53, with lower scores indicating greater impairment.
ADAS-Cog13, Alzheimer’s Disease Assessment Scale, 13-item;
ADCS-ADL-MCI, Alzheimer’s Disease Cooperative Study-Activities of Daily Living Inventory, mild cognitive impairment version;
ApoE, apolipoprotein E;
CDR-SB, Clinical Dementia Rating Scale-Sum of Boxes;
CI, confidence interval;
MMSE, Mini-Mental State Examination;
SE, standard error.

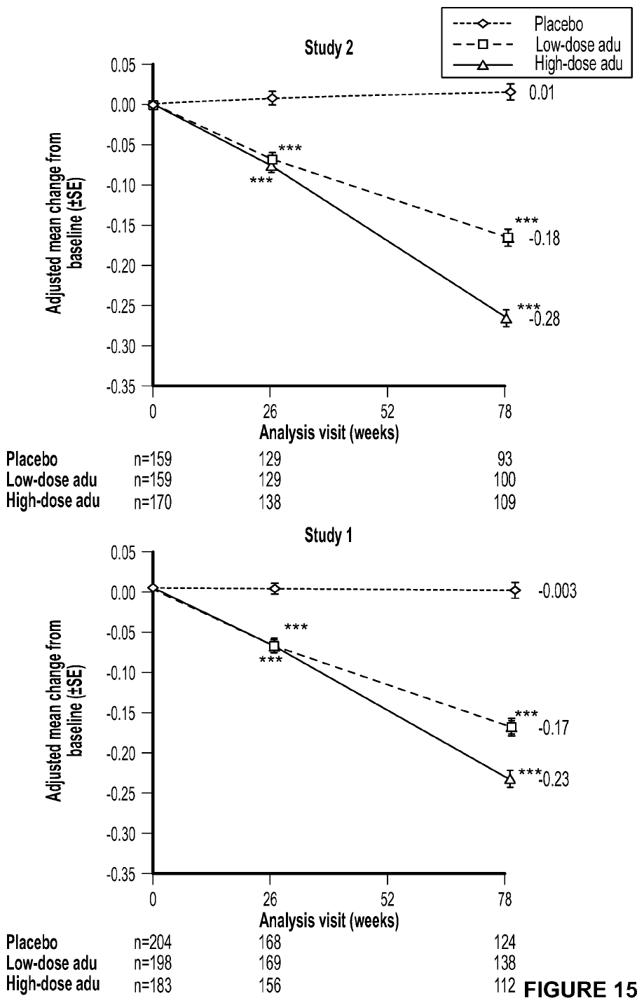

In the amyloid PET substudy, a dose- and time-dependent reduction in amyloid PET SUVR was observed at Week 78. The difference in adjusted mean change from baseline between high-dose aducanumab and placebo was -0.28 (95% confidence interval, −0.31 to −0.25; P<0.001) in Study 2 and −0.23 (95% confidence interval, −0.26 to −0.21; P<0.001) in Study 1 (FIG. 15).

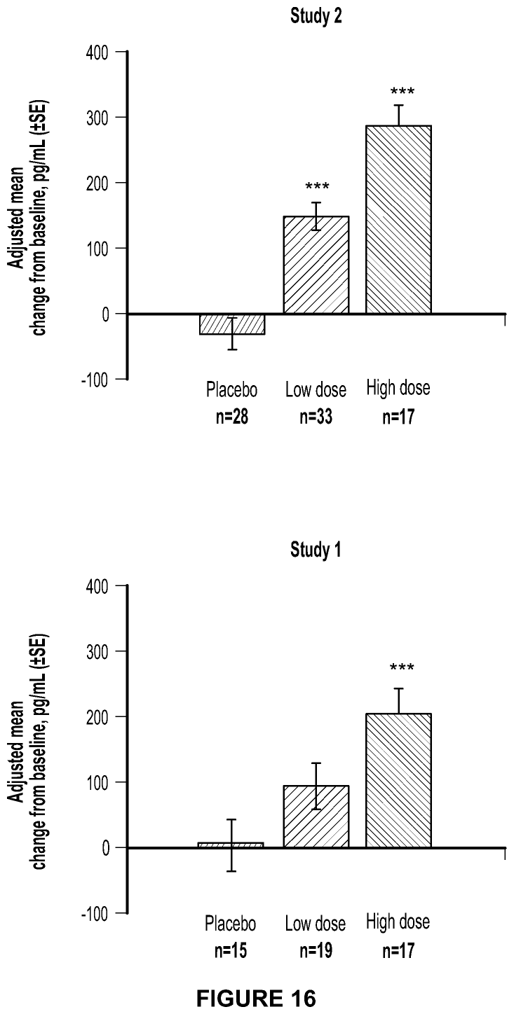

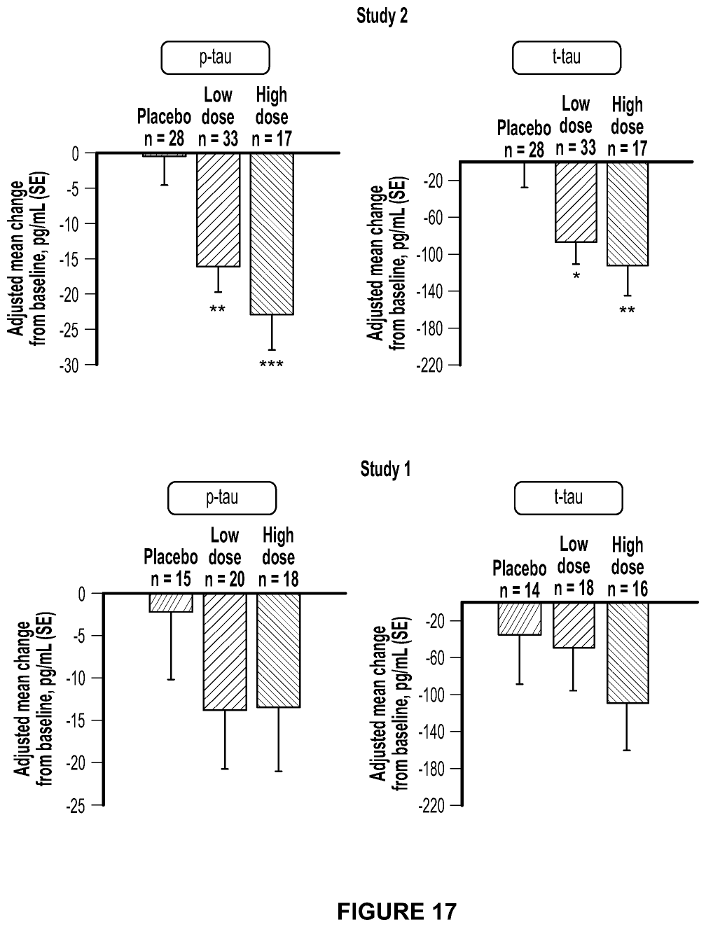

In the CSF substudy, there was a dose-dependent increase in CSF levels of Aβ₁₋₄₂, a measure of target engagement, in patients receiving high-dose aducanumab compared with those receiving placebo (FIG. 16). Difference in adjusted mean change from baseline versus placebo for CSF levels of Aβ₁₋₄₂ at Week 78 was 318.88 (95% confidence interval, 247.18 to 390.58; P<0.001) and 198.73 (95% confidence interval 91.02 to 306.43; P<0.001) for Study 2 and Study 1, respectively. As expected, CSF Aβ₁₋₄₀ did not differ significantly between treatment groups (data not shown). In Study 2, there was a dose-dependent and significant reduction in CSF levels of p-tau, a disease biomarker, and t-tau, a nonspecific marker of neurodegeneration (FIG. 17). In Study 1, there was a numerical reduction that did not reach statistical significance in the CSF levels of p-tau and t-tau.

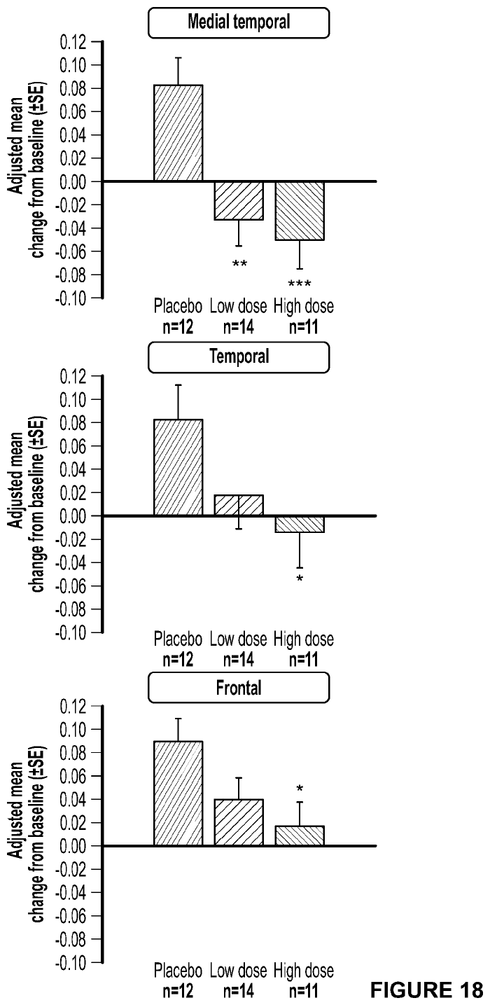

The tau PET substudy included patients pooled across both studies. Aducanumab-treated patients had statistically significant, dose-dependent reductions in tau PET SUVRs in the medial temporal, temporal, and frontal lobe versus placebo-treated patients (FIG. 18). Results from the parietal, cingulate, and occipital lobes were not statistically significant (data not shown).

To assess why results from Study 2 and Study 1 differed, analysis plans were developed that included multiple lines of investigation. The two main factors contributing to discordance in the results of the high-dose arms of Study 1 versus Study 2 were the influence of rapid progressors (defined as those patients with a change from baseline in CDR-SB score of >8 at Week 78) and lower dosing.

A subgroup of patients, denoted the post-PV4 subgroup, who consented to PV4 (or a later protocol version) on or prior to Week 16 was analyzed. Patients in this subgroup who randomized to receive high-dose aducanumab had the opportunity to receive the full course of fourteen 10-mg/kg doses of aducanumab. Protocol management of ARIA in this subgroup also specified fewer dosing interruptions and the ability to resume titration after a dosing interruption.

Therefore, the mean cumulative dose in the high-dose arm of the Study 1 pre-PV4 subgroup was lower compared with that of the post-PV4 subgroup (Table 8). In addition, 15 of the 18 rapid progressors in Study 1 were in the pre-PV4 subgroup.

Thus, analyses of the Study 1 post-PV4 subgroup illustrate results absent the influence of lower dosing and rapidly progressing patients. The difference from placebo in adjusted mean change from baseline in CDR-SB score at Week 78 was −0.49 (95% confidence interval, −1.02 to 0.04) for patients in the post-PV4 group of Study 1 receiving high-dose aducanumab, representing a 27% decrease (Table 8).

TABLE 8
Results for the post-PV4 subgroup of Study 1 at Week 78

| | | Difference vs. placebo (%)* | |
|---|---|---|---|
| | Placebo | 95% CI | |
| | decline ± SE | Low dose | High dose |
| Endpoint | (n = 251) | (n = 270) | (n = 288) |
| CDR-SB^{†} | 1.84 ± 0.20 | −0.42 (−23%) | −0.49 (−27%) |
| | | −0.94, 0.10 | −1.02, 0.04 |
| MMSE^{‡} | −3.8 ± 0.4 | 0.9 (−24%) | 0.5 (−13%) |
| | | 0.0, 1.9 | −0.4, 1.5 |
| ADAS-Cog 13^{§} | 4.85 ± 0.69 | −0.14 (−3%) | −0.98 (−20%) |
| | | −1.93, 1.66 | −2.83, 0.87 |
| ADCS-ADL-MCI^{¶} | −5.0 ± 0.7 | 1.5 (−30%) | 3.0 (−60%) |
| | | −0.2, 3.2 | 1.2, 4.7 |

The mean cumulative dose in the high-dose arm of the Study 1 prc-PV4 subgroup was 104 mg/kg, and the mean number of doses of 10 mg/kg was 6.5, compared with 130 mg/kg and 10.8, respectively, in the post-PV4 subgroup. Cumulative dose was defined as the sum of all doses received for each patient during the placebo-controlled period.
*Differcnce versus placebo at Week 78. Negative percentage means less progression in the treated arm.
^{†}CDR-SB scores range from 0 to 18, with higher scores indicating greater impairment.
^{‡}MMSE scores range from 0 to 30, with lower scores indicating greater impairment.
^{§}ADAS-Cog13 scores range from 0 to 85, with higher scores indicating greater impairment.
^{¶}ADCS-ADL-MCI scores range from 0 to 53, with lower scores indicating greater impairment.
ADAS-Cog13, Alzheimer's Disease Assessment Scale, 13-item;
ADCS-ADL-MCI, Alzheimer's Disease Cooperative Study—Activities of Daily Living Inventory, mild cognitive impairment version;
ApoE, apolipoprotein E;
CDR-SB, Clinical Dementia Rating Scale-Sum of Boxes;
CI, confidence interval;
MMSE, Mini-Mental State Examination;
PV4, protocol version 4;
SE, standard error.

### Conclusion

Analyses from the final data set in Study 2 demonstrated a statistically significant advantage of high-dose aducanumab over placebo on the prespecified primary endpoint, CDR-SB, at Week 78. A statistically significant slowing of clinical decline was detected across three secondary endpoints, demonstrating consistent clinical superiority of aducanumab over placebo using outcome measures that assess both cognitive and functional changes in patients with early AD.

In Study 1, the primary endpoint was not met. Post hoc analyses on a subset of patients (the post-PV4 subgroup) with 10 mg/kg aducanumab as the target dose suggested that the results of Study 1 were due to insufficient dosing and a cluster of patients with rapid disease progression.

Results from substudies of proximal pharmacodynamic biomarkers (Aβ CSF and PET) and downstream biomarkers specific to Alzheimer's disease (tau PET and CSF p-tau) and neurodegeneration (CSF t-tau) further support the clinical findings. This was the first demonstration in Phase 3 trials where modification of biomarkers of underlying disease pathology was associated with statistically significant slowing of clinical decline. It is important to consider the small number of patients in the tau PET and CSF substudies. However, consistent with the lower cumulative dose in Study 1, effects on biomarkers in the high-dose arm of Study 1 were smaller compared with Study 2.

The most common adverse event associated with aducanumab was ARIA-E, an imaging abnormality detected via brain MRI in both trials. As observed in other studies of anti-amyloid monoclonal antibodies, ARIA was mostly asymptomatic, and most patients with ARIA were able to continue treatment. Overall, the safety and tolerability profile of aducanumab in Study 2 and Study 1 was consistent with previous studies.

In summary, Study 2 demonstrates clinical superiority of aducanumab over placebo, reflecting an advantage over the current standard of care for patients with early Alzheimer's disease. Results from a subgroup of patients in Study 1 who were exposed to high-dose aducanumab support these findings.

### OTHER EMBODIMENTS

While the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method for treating Alzheimer's disease in a human subject in need thereof, the method comprising administering to the human subject multiple doses of an anti-beta-amyloid antibody, wherein the multiple doses are administered as follows:
(a) administering the anti-beta-amyloid antibody to the subject in an amount of 1 mg/kg of body weight of the subject;
(b) 4 weeks after step (a), administering the antibody to the subject in an amount of 1 mg/kg of body weight of the subject;
(c) 4 weeks after step (b), administering the antibody to the subject in an amount of 3 mg/kg of body weight of the subject;
(d) 4 weeks after step (c), administering the antibody to the subject in an amount of 3 mg/kg of body weight of the subject;
(e) 4 weeks after step (d), administering the antibody to the subject in an amount of 6 mg/kg of body weight of the subject;
(f) 4 weeks after step (e), administering the antibody to the subject in an amount of 6 mg/kg of body weight of the subject; and
(g) in consecutive intervals of 4 weeks after step (f), administering at least 15 doses of the antibody in an amount of 10 mg/kg of body weight of the subject,
wherein the anti-beta-amyloid antibody comprises a heavy chain variable region (VH) and a light chain variable region (VL),
wherein the VH comprises a complementarity determining region (VHCDR1) with the amino acid sequence of SEQ ID NO:3, a VHCDR2 with the amino acid sequence of SEQ ID NO:4, and a VHCDR3 with the amino acid sequence of SEQ ID NO:5, and
wherein the VL comprises a VLCDR1 with the amino acid sequence of SEQ ID NO:6, a VLCDR2 with the amino acid sequence of SEQ ID NO:7, and a VLCDR3 with the amino acid sequence of SEQ ID NO:8.

2. The method of claim 1, wherein step (g) comprises administering at least 18 doses of the antibody, in consecutive intervals of 4 weeks, each in an amount of 10 mg/kg of body weight of the subject.

3. The method of claim 1, wherein step (g) comprises administering at least 20 doses of the antibody, in consecutive intervals of 4 weeks, each in an amount of 10 mg/kg of body weight of the subject.

4. The method of any one of claims 1 to 3, wherein all of the doses specified in steps (a)-(g) are administered without interruption even if the human subject develops an Amyloid Related Imaging Abnormality (ARIA) during the course of treatment.

5. The method of any one of claims 1 to 3, wherein the human subject develops an ARIA during the course of treatment and all of the doses specified in steps (a)-(g) are administered without interruption.

6. The method of any one of claims 1 to 5, wherein the Alzheimer's disease is mild Alzheimer's disease, early Alzheimer's disease, prodromal Alzheimer's disease, mild Alzheimer's disease dementia, or mild cognitive impairment due to Alzheimer's disease.

7. A method for treating mild Alzheimer's disease, early Alzheimer's disease, prodromal Alzheimer's disease, mild Alzheimer's disease dementia, or mild cognitive impairment due to Alzheimer's disease in a human subject in need thereof, the method comprising administering to the human subject multiple doses of an anti-beta-amyloid antibody, wherein the method comprises administering in consecutive intervals of 4 weeks at least 6 doses of the antibody,
wherein each dose is in an amount of 10 mg/kg of body weight of the subject, wherein the anti-beta-amyloid antibody comprises a heavy chain variable region (VH) and a light chain variable region (VL),
wherein the VH comprises a complementarity determining region (VHCDR1) with the amino acid sequence of SEQ ID NO:3, a VHCDR2 with the amino acid sequence of SEQ ID NO:4, and a VHCDR3 with the amino acid sequence of SEQ ID NO:5, and
wherein the VL comprises a VLCDR1 with the amino acid sequence of SEQ ID NO:6, a VLCDR2 with the amino acid sequence of SEQ ID NO:7, and a VLCDR3 with the amino acid sequence of SEQ ID NO:8.

8. The method of claim 7, wherein the method comprises administering in consecutive intervals of 4 weeks at least 8 doses of the antibody, wherein each dose is in an amount of 10 mg/kg of body weight of the subject.

9. The method of claim 7, wherein the method comprises administering in consecutive intervals of 4 weeks at least 10 doses of the antibody, wherein each dose is in an amount of 10 mg/kg of body weight of the subject.

10. The method of claim 7, wherein the method comprises administering in consecutive intervals of 4 weeks at least 15 doses of the antibody, wherein each dose is in an amount of 10 mg/kg of body weight of the subject.

11. The method of any one of claims 7 to 10, wherein all of the doses specified are administered without interruption even if the human subject develops an ARIA during the course of treatment.

12. The method of any one of claims 7 to 10, wherein the human subject develops an ARIA during the course of treatment and all of the doses specified are administered without interruption.

13. A method for treating Alzheimer's disease in a human subject in need thereof, the method comprising administering to the human subject multiple doses of an anti-beta-amyloid antibody, wherein the multiple doses are administered as follows:
(a) administering the anti-beta-amyloid antibody to the subject in an amount of 1 mg/kg of body weight of the subject;
(b) 4 weeks after step (a), administering the antibody to the subject in an amount of 1 mg/kg of body weight of the subject;
(c) 4 weeks after step (b), administering the antibody to the subject in an amount of 3 mg/kg of body weight of the subject;
(d) 4 weeks after step (c), administering the antibody to the subject in an amount of 3 mg/kg of body weight of the subject.
(e) 4 weeks after step (d), administering the antibody to the subject in an amount of 6 mg/kg of body weight of the subject;
4 weeks after step (e), administering the antibody to the subject in an amount of 6 mg/kg of body weight of the subject; and
(g) in consecutive intervals of 4 weeks after step (f), administering the antibody to the subject in an amount of 10 mg/kg of body weight of the subject,
wherein the anti-beta-amyloid antibody comprises a heavy chain variable region (VH) and a light chain variable region (VL),
wherein the VH comprises a complementarity determining region (VHCDR1) with the amino acid sequence of SEQ ID NO:3, a VHCDR2 with the amino acid sequence of SEQ ID NO:4, and a VHCDR3 with the amino acid sequence of SEQ ID NO:5,
wherein the VL comprises a VLCDR1 with the amino acid sequence of SEQ ID NO:6, a VLCDR2 with the amino acid sequence of SEQ ID NO:7, and a VLCDR3 with the amino acid sequence of SEQ ID NO:8, and
wherein all of the doses specified are administered without interruption even if the human subject develops an ARIA during the course of treatment.

14. The method of claim 13, wherein the human subject develops an ARIA during the course of treatment and all of the doses specified are administered without interruption.

15. The method of claim 13 or 14, wherein step (g) comprises administering at least 6 doses of the antibody, in consecutive intervals of 4 weeks, each in an amount of 10 mg/kg of body weight of the subject.

16. The method of claim 13 or 14, wherein step (g) comprises administering at least 8 doses of the antibody, in consecutive intervals of 4 weeks, each in an amount of 10 mg/kg of body weight of the subject.

17. The method of claim 13 or 14, wherein step (g) comprises administering at least 10 doses of the antibody, in consecutive intervals of 4 weeks, each in an amount of 10 mg/kg of body weight of the subject.

18. The method of any one of claims 13 to 17, wherein the Alzheimer's disease is mild Alzheimer's disease, early Alzheimer's disease, prodromal Alzheimer's disease, mild Alzheimer's disease dementia, or mild cognitive impairment due to Alzheimer's disease.

19. The method of any one of the preceding claims, wherein each administration is performed intravenously.

20. A method for treating Alzheimer's disease in a human subject in need thereof, the method comprising administering to the human subject multiple doses of an anti-beta-amyloid antibody, wherein the multiple doses are administered as follows:
(a) administering intravenously the anti-beta-amyloid antibody to the subject in an amount of 1 mg/kg of body weight of the subject;
(b) 4 weeks after step (a), administering intravenously the antibody to the subject in an amount of 1 mg/kg of body weight of the subject;
(c) 4 weeks after step (b), administering intravenously the antibody to the subject in an amount of 3 mg/kg of body weight of the subject;
(d) 4 weeks after step (c), administering intravenously the antibody to the subject in an amount of 3 mg/kg of body weight of the subject;
(e) 4 weeks after step (d), administering intravenously the antibody to the subject in an amount of 6 mg/kg of body weight of the subject;
4 weeks after step (e), administering intravenously the antibody to the subject in an amount of 6 mg/kg of body weight of the subject; and
(g) in consecutive intervals of 4 weeks after step (f), administering intravenously at least 6 doses of the antibody in an amount of 10 mg/kg of body weight of the subject,
wherein the anti-beta-amyloid antibody comprises a heavy chain variable region (VH) and a light chain variable region (VL),
wherein the VH comprises a complementarity determining region (VHCDR1) with the amino acid sequence of SEQ ID NO:3, a VHCDR2 with the amino acid sequence of SEQ ID NO:4, and a VHCDR3 with the amino acid sequence of SEQ ID NO:5, and
wherein the VL comprises a VLCDR1 with the amino acid sequence of SEQ ID NO:6, a VLCDR2 with the amino acid sequence of SEQ ID NO:7, and a VLCDR3 with the amino acid sequence of SEQ ID NO:8.

21. The method of claim 20, wherein step (g) comprises administering intravenously at least 8 doses of the antibody, in consecutive intervals of 4 weeks, each in an amount of 10 mg/kg of body weight of the subject.

22. The method of claim 20, wherein step (g) comprises administering intravenously at least 10 doses of the antibody, in consecutive intervals of 4 weeks, each in an amount of 10 mg/kg of body weight of the subject.

23. The method of any one of claims 20 to 22, wherein all of the doses specified in steps (a)-(g) are administered without interruption even if the human subject develops an ARIA during the course of treatment.

24. The method of any one of claims 20 to 22, wherein the human subject develops an ARIA during the course of treatment and all of the doses specified in steps (a)-(g) are administered without interruption.

25. The method of any one of claims 20 to 24, wherein the Alzheimer's disease is mild Alzheimer's disease, early Alzheimer's disease, prodromal Alzheimer's disease, mild Alzheimer's disease dementia, or mild cognitive impairment due to Alzheimer's disease.

26. The method of any one of the preceding claims, wherein the human subject is confirmed to have a brain amyloid beta pathology prior to the initiation of treatment.

27. The method of claim 26, wherein the brain amyloid beta pathology is determined by positron emission tomography (PET) imaging.

28. The method of apv one of claims 1 to 27. wherein:
the VH comprises the amino acid sequence of SEQ ID NO:1; and
the VL comprises the amino acid sequence of SEQ ID NO:2.

29. The method of any one of claims 1 to 28, wherein the antibody comprises a human IgG1 constant region.

30. The method of any one of claims 1 to 27, wherein the anti-beta-amyloid antibody comprises a heavy chain and a light chain, wherein:
the heavy chain comprises the amino acid sequence of SEQ ID NO:10; and
the light chain comprises the amino acid sequence of SEQ ID NO:11.

31. A method of treating Alzheimer's disease in a human subject in need thereof, the method comprising administering to the human subject a therapeutically effective amount of an anti-beta-amyloid antibody comprising a heavy chain variable region (VH) and a light chain variable region (VL), wherein the VH comprises a VH complementarity determining region 1 (VHCDR1) with the amino acid sequence of SEQ ID NO:3, a VHCDR2 with the amino acid sequence of SEQ ID NO:4, and a VHCDR3 with the amino acid sequence of SEQ ID NO:5, and wherein the VL comprises a VLCDR1 with the amino acid sequence of SEQ ID NO:6, a VLCDR2 with the amino acid sequence of SEQ ID NO:7, and a VLCDR3 with the amino acid sequence of SEQ ID NO:8, wherein the human subject has p-tau tangles, p-tau threads, and/or p-tau neuritic plaques, optionally wherein the human subject has neocortical p-tau tangles, neocortical p-tau threads, and/or neocortical p-tau neuritic plaques.

32. The method of claim 31, wherein the administration of the anti-beta-amyloid antibody reduces p-tau tangles, p-tau threads, and/or p-tau neuritic plaques in the brain of the human subject or the amount of phosphorylated tau (p-tau) and/or total tau (t-tau) in the cerebrospinal fluid (CSF) of the human subject.

33. The method of claim 31 or 32, wherein, prior to the administration of the anti- beta-amyloid antibody, p-tau tangles, p-tau threads, and/or p-tau neuritic plaques are detected by positron emission tomography (PET) scanning of the human subject's brain or by analysis of the amount of p-tau and/or t-tau in the human subject's CSF.

34. The method of any one of claims 31 to 33, wherein the method further comprises monitoring during treatment p-tau tangles, p-tau threads, and/or p-tau neuritic plaques by PET scanning of the human subject's brain or by analysis of the amount of p-tau and/or t-tau in the human subject's CSF.

35. The method of any one of claims 31 to 33, wherein the amount of the anti-beta-amyloid antibody administered and/or frequency of administration of the anti-beta-amyloid antibody is adjusted during treatment by monitoring p-tau tangles, p-tau threads, and/or p-tau neuritic plaques in the human subject's brain using PET scanning, or by analysis of the amount of p-tau and/or t-tau in the human subject's CSF.

36. The method of claim 35, wherein the treatment results in (i) a reduction in SUVR, density, and/or distribution of p-tau tangles, p-tau threads, and/or p-tau neuritic plaques relative to a prior PET scan, or (ii) a reduction in the amount of p-tau and/or t-tau in a CSF analysis relative to a prior CSF analysis.

37. A method of reducing tau in a human subject having Alzheimer's disease, the method comprising administering to the human subject an effective amount of an anti-beta- amyloid antibody comprising a VH and a VL, wherein the VH comprises a VHCDR1 with the amino acid sequence of SEQ ID NO:3, a VHCDR2 with the amino acid sequence of SEQ ID NO:4, and a VHCDR3 with the amino acid sequence of SEQ ID NO:5, and wherein the VL comprises a VLCDR1 with the amino acid sequence of SEQ ID NO:6, a VLCDR2 with the amino acid sequence of SEQ ID NO:7, and a VLCDR3 with the amino acid sequence of SEQ ID NO:8.

38. A method of treating Alzheimer's disease by reducing the amount of tau in a human subject in need thereof, the method comprising administering to the human subject an effective amount of an anti-beta-amyloid antibody comprising a VH and a VL, wherein the VH comprises a VHCDR1 with the amino acid sequence of SEQ ID NO:3, a VHCDR2 with the amino acid sequence of SEQ ID NO:4, and a VHCDR3 with the amino acid sequence of SEQ ID NO:5, and wherein the VL comprises a VLCDR1 with the amino acid sequence of SEQ ID NO:6, a VLCDR2 with the amino acid sequence of SEQ ID NO:7, and a VLCDR3 with the amino acid sequence of SEQ ID NO:8.

39. The method of claim 37 or 38, wherein the human subject is, or has previously been, diagnosed as having p-tau tangles, p-tau threads, and/or p-tau neuritic plaques in the brain and/or an increased amount of p-tau and/or t-tau in the human subject's CSF relative to a human subject without Alzheimer's disease.

40. The method of any one of claims 37 to 39, wherein the amount of tau in the brain and/or the CSF of the human subject is reduced.

41. The method of any one of claims 37 to 40, wherein the amount of p-tau and/or t- tau in the human subject is reduced.

42. The method of any one of claims 37 to 41, wherein the human subject has elevated levels of tau, prior to administration of the anti-beta-amyloid antibody, as measured in the CSF or in the brain by PET scanning.

43. The method of any one of claims 31 to 42, wherein the Alzheimer's disease is mild Alzheimer's disease, early Alzheimer's disease, prodromal Alzheimer's disease, mild Alzheimer's disease dementia, mild cognitive impairment due to Alzheimer's disease, mid-stage Alzheimer's disease, or late-stage Alzheimer's disease, optionally wherein mid-stage Alzheimer's disease is characterized by a Mini-Mental State Examination (MMSE) score of about 10-20 or equivalent score on other scales and late-stage Alzheimer's disease is characterized by an MMSE score of about 9 or less or equivalent score on other scales.

44. The method of any one of claims 31 to 42, wherein the Alzheimer's disease is mild cognitive impairment due to Alzheimer's disease.

45. The method of any one of claims 31 to 42, wherein the Alzheimer's disease is mild Alzheimer's disease dementia.

46. The method of any one of claims 31 to 45, wherein the VH comprises the amino acid sequence of SEQ ID NO:1 and the VL comprises the amino acid sequence of SEQ ID NO:2.

47. The method of any one of claims 31 to 45, wherein the anti-beta-amyloid antibody comprises a heavy chain comprising the amino acid sequence of SEQ ID NO:10 and a light chain comprising the amino acid sequence of SEQ ID NO:11.

48. The method of any one of claims 31 to 47, wherein the anti-beta-amyloid antibody is administered intravenously.

49. The method of any one of claims 31 to 48, comprising administering the anti-beta-amyloid antibody in an amount of 3 mg antibody/kg of body weight of the human subject.

50. The method of any one of claims 31 to 48, comprising administering the anti-beta-amyloid antibody in an amount of 6 mg antibody/kg of body weight of the human subject.

51. The method of any one of claims 31 to 48, comprising administering the anti-beta-amyloid antibody in an amount of 10 mg antibody/kg of body weight of the human subject.

52. The method of any one of claims 31 to 48, comprising administering the anti-beta-amyloid antibody in multiple doses as follows:
(a) administering the anti-beta-amyloid antibody to the human subject in an amount of 1 mg antibody/kg of body weight of the human subject;
(b) 4 weeks after step (a), administering the antibody to the human subject in an amount of 1 mg antibody/kg of body weight of the human subject;
(c) 4 weeks after step (b), administering the antibody to the human subject in an amount of 3 mg antibody/kg of body weight of the human subject:
(d) 4 weeks after step (c), administering the antibody to the human subject in an amount of 3 mg antibody/kg of body weight of the human subject;
(e) 4 weeks after step (d), administering the antibody to the human subject in an amount of 6 mg antibody/kg of body weight of the human subject;
4 weeks after step (e), administering the antibody to the human subject in an amount of 6 mg antibody/kg of body weight of the human subject; and
(g) in consecutive intervals of 4 weeks after step (f), administering the antibody to the human subject in an amount of 10 mg antibody/kg of body weight of the human subject.

53. The method of any one of claims 31 to 48, comprising administering the antibody at a cumulative dose of at least 150 mg antibody/kg of body weight of the human subject.

54. The method of any one of claims 31 to 48, comprising administering the antibody at a cumulative dose of at least 200 mg antibody/kg of body weight of the human subject.

55. The method of any one of claims 31 to 48, comprising administering the antibody in an amount of 10 mg antibody/kg of body weight of the human subject every 4 weeks over at least 52 weeks.

56. The method of any one of claims 31 to 48, comprising administering the antibody in an amount of 6 mg antibody/kg of body weight of the human subject every 4 weeks over at least 112 weeks.

57. The method of any one of claims 31 to 48, comprising administering the antibody to the human subject in multiple doses and wherein the multiple doses comprise:
(a) at least two doses of 3 mg antibody/kg of body weight of the human subject every 4 weeks; and
(b) at least 30 doses of 6 mg antibody/kg of body weight of the human subject every 4 weeks.

58. The method of any one of claims 31 to 57, wherein the human subject is an ApoE3 carrier.

59. The method of any one of claims 31 to 58, wherein the human subject does not develop an Amyloid Related Imaging Abnormality (ARIA) during the course of treatment that requires suspension of treatment.
